(19) United States
(12) Patent Application Publication
(10) Pub. No.: US 2024/0316243 A1
(43) Pub. Date: Sep. 26, 2024

(54) METHODS AND MATERIAL COMPOSITIONS FOR SEALING A TRACT

(71) Applicant: PneumoNix Medical, Inc., Brooklyn, NY (US)

(72) Inventors: Shashwat Gupta, Baltimore, MD (US); Andrew Eisenthal, III, Baltimore, MD (US); Joseph White, Baltimore, MD (US); Stephen Spiegelberg, Baltimore, MD (US); Jamie Robertson, Blatimore, MD (US)

(73) Assignee: PneumoNIX Medical, Inc., Brooklyn, NY (US)

(21) Appl. No.: 18/290,042

(22) PCT Filed: May 11, 2022
(86) PCT No.: PCT/US2022/028756

§ 371 Date: Nov. 9, 2023

Related U.S. Application Data
(60) Provisional application No. 63/187,669, filed on May 12, 2021.

(51) Int. Cl. A61L 24/04; A61B 17/00; A61L 24/00

(52) U.S. Cl. CPC A61L 24/046; A61B 17/0057; A61L 24/001; A61L 2400/06; A61L 2430/36

(57) ABSTRACT

This disclosure relates to devices, method, and material compositions. Specifically, the present invention related to a delivery device to deliver a material composition that seals a tract, for example during a percutaneous intervention.

### RELATED APPLICATIONS

This application claims priority to U.S. Ser. No. 63/187,669 filed on May 12, 2021, which is hereby incorporated into this disclosure in its entirety.

### FIELD OF THE DISCLOSURE

The disclosure relates to devices, method, and material compositions. Specifically, the present invention related to a delivery device to deliver a material composition that seals a tract, for example during a percutaneous intervention.

### BACKGROUND

Lung cancer is currently the leading cause of cancer death among both men and women. Accounting for 155,000 deaths per year in the U.S, this number is expected to rise especially in third world countries due to high pollution levels and high rates of smoking.

The anatomy of pulmonary system is shown in FIG. 1.

The lungs which sit on opposite sides of the heart can be distinguished by the number of lobes. The right lung is divided into three lobes while the left lung has two lobes. The lines which divide each lobe are known as fissures and are oriented at off angles to one-another causing the lobes to overlap each other.

Air is inhaled through the trachea and splits into the right and left lung at the bifurcation, better known as the carina. These smaller air pathways are bronchi which then evolve into secondary and tertiary bronchi, defined by smaller and smaller passageways. Towards the end of the air passageway the structure is referred to as a bronchiole which transports air to the final structure called the alveolar duct which comprises millions of alveoli facilitating gas exchange with the pulmonary vasculature.

The thoracic cavity, which houses the lungs, is delineated by the ribs ventrally, dorsally, and laterally. Between the ribs are intercostal muscles, which have fibers oriented in several different planes to aid in breathing. The external intercostal muscles aid in forced inhalation, as they help bend the ribs open to expand the transverse dimensions of the thoracic cavity. The internal intercostal muscles are responsible for the depression of the ribs, bending them inward thus decreasing the transverse dimensions of the thoracic cavity, aiding forced exhalation.

The lungs are enveloped in a thin serous membrane that dips into the fissures between the lobes, called the visceral pleura. This contiguous membrane is then reflected onto the outer aspect of the thoracic cavity (the innermost portion of the chest wall), called the parietal pleura, which is thicker than the visceral pleura. The space between the parietal and visceral pleura is called the pleural space, and is a potential space under normal physiologic conditions. The pleural space contains a serous fluid which aids the two layers in cushioning and sliding relative to one another. Additionally, the pleural space is a negative pressure space. Contraction of the diaphragm increases the volume of the thoracic cavity, thus creates a negative pressure within the pleural space causing the lungs to expand resulting in passive exhalation and active inhalation.

Solitary pulmonary nodule biopsy methods.

There are several methods to determine if lung cancer is present. These include serial imaging, sputum cytology, or tissue sample (biopsy). Biopsies can occur in several ways such as bronchoscopy, mediastinoscopy, and image guided biopsy.

Bronchoscopy is a procedure which allows a doctor to look at your airways through a thin instrument called a bronchoscope. The bronchoscope comprises either a flexible or rigid tube which has a visualization element on the end such as a fiber optic camera. The most common type of bronchoscope used for diagnosing lung lesions is an Endobronchial Ultrasound (EBUS). This device comprises a small balloon with an ultrasound element and biopsy needle at the tip. Via the ultrasound, the doctor can locate the lung lesion and retrieve a sample using the needle. Some problems with EBUS include poor diagnostic yield from the needle and an inability to sample peripheral lesions due to size constraints of the device and bronchi.

Mediastinoscopy is a procedure to look at the mediastinum, the area between and in front of the lungs. During this procedure a small incision is made in the sternal notch or on the left side of the chest next to the sternum. A small scope is inserted allowing tissue biopsy to be collected via the scope. The rate of this procedure has decreased rapidly due to the rise of EBUS and image guided biopsies such as computed tomography (CT).

During CT-guided biopsy the physician is able to direct the percutaneous biopsy device using constant imaging. The biopsy retrieved during this procedure can be either a fine needle or core biopsy, core being favored by pathologists over the fine-needle biopsies retrieved during EBUS. This is because it's easier to determine malignancy because the tissue architecture is maintained and the sample is larger. The number one complication during CT-guided lung biopsy is a pneumothorax which occurs 25-35% of the time. Furthermore, 5-15% of all CT-guided lung biopsy cases result in further observation or hospitalization due to pneumothorax. The definitive treatment for a pneumothorax is a chest tube to help re-expand the lung. This causes pain for the patient and increased costs for the healthcare system.

A pneumothorax, also called a collapsed lung, is defined as the entry of air into the pleural space. During a CT-guided biopsy a tract is made both through the chest wall and the lung parenchyma leading to the suspected lesion. Due to the inherent negative pressure of the pleural space, air from both outside the body and inside the lung will try and create equilibrium by filling the pleural space. When too much air enters this space an outside pressure is placed on the lung causing it to collapse (pneumothorax). To diagnose a pneumothorax a physician will perform a CT-scan or X-Ray immediately after the biopsy, 2 hours after the biopsy, and/or 4 hours after the biopsy. If the physician believes the patient is no longer at risk of a pneumothorax after imaging, the patient is sent home.

Although the physician cannot definitively determine which patients will develop a pneumothorax from a biopsy, there is a general consensus about which patient population and lesion type have increased risk of pneumothorax. For example, patients with emphysema are at higher risk of a pneumothorax. Emphysema causes the alveoli within the lungs to become damaged eventually weakening and rupturing. This causes larger air spaces and reduces the overall surface area of the lung thereby reducing the amount of oxygen that reaches the blood stream. When exhaling, the damaged alveoli don't exchange oxygen properly and air becomes trapped leaving no room for fresh oxygen-rich air to enter. The outer periphery of the lung can become so weak and trapped with air that a bullae can form. The trapped air in the periphery of the lungs and bullae acts as a source of air when crossed with a biopsy device and therefore contributes to the higher rate of pneumothorax in emphysematous patients.

Other factors which contribute to higher rates of pneumothorax are needle angle, needle path length, and trans-fissure lesions. During the procedure the physician will try and maintain a perpendicular path relative to the bronchial tree which minimizes the amount of the bronchiole that is crossed by the biopsy device. Additionally, the physician will try and take the shortest path possible without crossing fissures. As discussed previously, the right lung has three fissures defining the lobes while the left lung has two. Because each lobe is covered by a discrete visceral pleura layer avoiding a fissure is a necessary precaution. Rates of pneumothorax are higher as more pleural surfaces are crossed because it is creating more sources of entry for air.

The techniques currently employed either involve plugging the biopsy tract or attempting to remove excess air. For example, some physicians attempt to use a saline or blood patch. In this method saline or blood is injected post-biopsy into the needle tract. These fluids act as a barrier to air entering into the pleural space. Another technique is called air aspiration which uses a syringe to suck air from the pleural space post-biopsy relying on the internal physiological healing mechanisms to seal the tract.

There is currently one FDA approved device on the market with an indication to prevent pneumothorax during computed tomography (CT)-guided lung biopsy. The device which is commercialized under the name BioSentry™ utilizes a desiccated PEG-hydrogel plug which is inserted post-procedurally. The plug is hydrated from both fluids inside the lung tissue and a saline drop applicator. The plug has a limited volumetric expansion and limited length. In addition, many pneumothoraces occur upon immediate entry into the pleural space and entry into the lung parenchyma with the biopsy device. Therefore, there is a need for a device to prevent air from entering the pleural space immediately upon entry of the biopsy device into the pleural space (e.g., across one or more pleural membrances) and lung parenchyma, preferably while not interrupting the tissue collection process and being configured to be compatible with standard biopsy tools.

One aspect of the invention relates to temperature sensitive polymers such as poloxamers, also known as pluronics or tri-block polymers. Poloxamers have a tri-block copolymer structure of hydrophilic polyethylene oxide (PEO) end blocks and a central hydrophobic polypropylene oxide (PPO) block resulting in a temperature sensitive polymer. At low temperatures, a mixture of poloxamer and water exists in the solution state and as the temperature of the solution is raised, a micellular structure is thermodynamically favored. As temperatures rises, the association of the poloxamer micelles serve to physically crosslink the polymer by forming a crystalline structure thereby resulting in gelation. Poloxamers have an established history in implantable medical devices such as temporary endovascular embolics (see U.S Patent 2005/0008610).

It is an object of this invention to control fluid flow during a surgical procedure, more preferably preventing pneumothorax during a lung intervention such as a biopsy, ablation, or excision. The invention generally includes the use of a delivery device used to deliver a material including but not limited to hydrogels, sealants, polymers, and thermosensitive polymers. A thermosensitive polymer is a polymer that can change from a liquid-gel state dependent on a specific temperature or a range of temperatures. In the present invention the thermosensitive polymer can be delivered inside the body in a liquid form at which it will transform to the gel state or it can be delivered in a gel state in which it will stay in a gel state inside the body.

### SUMMARY OF THE DISCLOSURE

One aspect of the present invention relates to a method of preventing pneumothorax comprising the steps of: (a) identifying a target site in the lung (b) advancing a device towards target site (c) administering a first composition comprising a polymer to act as a sealant, preferably a thermosensitive polymer, to a location at or before reaching the target site (d) further advancing the device to the target site (e) performing a surgical step at the surgical site using the device wherein the surgical step includes but is not limited to (biopsy specimen collection, tissue excision, thermal ablation) (f) removing the device upon completion of the surgical step while injecting more polymer material preferably a thermosensitive polymer wherein the thermosensitive polymer in both steps (c) and (f) act to prevent flow of a biologic fluid, more specifically the polymers and/or thermosensitive polymer act to prevent pneumothorax.

In certain embodiments, the present invention relates to the aforementioned method, wherein said thermosensitive is a block polymer or a branched copolymer.

In certain embodiments, the present invention relates to the aforementioned method, wherein said thermosensitive polymer is an optionally purified poloxamer, poloxamine, or pluronic.

In certain embodiments, the present invention relates to the aforementioned method, wherein said thermosensitive polymer is optionally purified and selected from the group consisting of poloxamine 1107, poloxamine 1307, poloxamer 338 and poloxamer 407.

In certain embodiments, the present invention relates to the aforementioned method, wherein said thermosensitive polymer solution has a transition temperature of between about 5° C. and 40° C., more preferably the thermosensitive polymer has a transition temperature between about 5° C. and 15° C.

In certain embodiments, the present invention relates to the aforementioned method, wherein the distance between the first composition and the ultimate target site is between about 0 to 15 cm, 1 cm to 10 cm, 2 cm to 4 cm. Artisans within the art will appreciate that any distance within these bounds and values outside of these bounds have been contemplated and appreciated.

In certain embodiments, the present invention relates to the aforementioned method, wherein the first composition is administered through a device including but not limited to a coaxial biopsy device, percutaneous access device, dual lumen catheter, triple lumen catheter, and ablation device.

In certain embodiments, the present invention relates to the aforementioned method, wherein the first composition is administered to a site proximal to a target site wherein the proximal site is the parietal pleura, visceral pleura, pleural space, lung parenchyma, lung fissure, the inner/outer surface of the parietal pleura, the inner/outer surface of the visceral pleura. It has been recognized that the first composition can be administered at or proximal to the target site as the device is advancing to the target site and when the device is being removed from the target site.

In certain embodiments, the present invention relates to the aforementioned method, wherein the target site is selected from the group consisting of but not limited to a lung, brain, kidney, liver, spleen, lesion, hemorrhage, tumor, dural sac, cancerous tissue, organ, and aneurysm.

In certain embodiments, the present invention relates to the aforementioned method, wherein the surgical step or surgical procedure is selected from the group consisting of but not limited to a biopsy, tissue excision, lavage, aspiration, ablation, resection, wedge resection, and VATS (video-assisted thoracoscopic surgery).

In certain embodiments, the present invention relates to the aforementioned method, wherein said first composition is injected via a catheter or syringe that has a volume capacity between 0.25 mL to 10 mL or 1 mL to 3 mL. Artisans within the art will appreciated that any volume within these bounds and volumes outside of these bounds have been contemplated and appreciated.

In certain embodiments, the present invention relates to the aforementioned method, wherein the first composition comprises a thermosensitive polymer that is administered in a gel state. Artisans within the art will appreciate that due to the thermosensitive nature the first composition can undergo a liquid-gel, liquid-liquid, gel-liquid, or gel-gel transition depending on the temperature that the first composition is exposed to, all of these transformations have been contemplated and appreciated.

In certain embodiments, the present invention relates to the aforementioned method, wherein the first composition when administered in the body and preferably proximal to or at a target site helps to prevent the flow of biologic fluid including but not limited to air (internal and external to lungs), blood, and tumor cells.

In certain embodiments, the present invention relates to the aforementioned method, wherein said first composition comprises about 5% to 45% or 10% to 40% of said thermosensitive polymer.

In certain embodiments, the present invention relates to the aforementioned method, wherein the thermosensitive polymer further comprises a contrast-enhancing agent and/or a tissue dye material.

In certain embodiments, the present invention relates to the aforementioned method, wherein said contrast-enhancing agent is selected from the group consisting of radiopaque materials, heavy atoms, transition metals, dyes, and radionuclide-containing materials.

In certain embodiments, the present invention relates to the aforementioned method, wherein the composition comprises about 5% to 45% or 10% to 40% of said thermosensitive polymer and between about 0% to 25% contrast-enhancing agent. Furthermore, a dye can also be included in the composition.

In certain embodiments, the present invention relates to the aforementioned method, wherein said thermosensitive polymer is optionally a poloxamer or poloxamine; wherein said thermosensitive polymer has a transition temperature between about 5° C. and 15° C.; said delivery device is a coaxial biopsy or percutaneous access device; said site where the thermosensitive polymer is administered is proximal to the target site; wherein the target site is located within a lung, parietal pleura, visceral pleura, pleural space, or lung fissure; wherein the surgical step administered to the target site is a biopsy and tissue excision; wherein the thermosensitive polymer is administered via a syringe in a gel state and optionally contains a contrast-enhancing agent and optionally contains a tissue dye; wherein the thermosensitive polymer prevents the flow of biologic fluid such as air (internal and external to lungs), blood, and tumor cells; wherein the thermosensitive polymer can also optionally be administered to the tissue and the device is being removed from the target site.

### BRIEF DESCRIPTION OF THE DRAWINGS

FIG. 1 illustrates the anatomy of the pulmonary system

FIG. 2 illustrates the anatomy of the pulmonary system specifically the pleural space

FIG. 3A illustrates a device adapted to deploy a first composition.

FIG. 3B illustrates a device adapted to deploy a first composition.

FIG. 3C illustrates a device adapted to deploy a first composition.

FIG. 3D illustrates a device adapted to deploy a first composition.

FIG. 3E illustrates a device adapted to deploy a first composition.

FIG. 3F illustrates a device adapted to deploy a first composition.

FIG. 4A illustrates the device of FIGS. 3A-3F deploying a first composition in a procedure.

FIG. 4B illustrates the device of FIGS. 3A-3F deploying a first composition in a procedure.

FIG. 4C illustrates the device of FIGS. 3A-3F deploying a first composition in a procedure.

FIG. 4D illustrates the device of FIGS. 3A-3F deploying a first composition in a procedure.

FIG. 5A illustrates an alternative device to deploy a first composition in a procedure.

FIG. 5B illustrates an alternative device to deploy a first composition in a procedure.

FIG. 6A illustrates the use of the device and polymer to prevent pneumothorax in an in-vivo swine model.

FIG. 6B illustrates the use of the device and polymer to prevent pneumothorax in an in-vivo swine model.

FIG. 6C illustrates the use of the device and polymer to prevent pneumothorax in an in-vivo swine model.

FIG. 6D illustrates the use of the device and polymer to prevent pneumothorax in an in-vivo swine model.

FIG. 6E illustrates the use of the device and polymer to prevent pneumothorax in an in-vivo swine model.

### DETAILED DESCRIPTION

As used herein, the singular forms “a”, “an”, and “the” refer to one or more than one, unless the context clearly dictates otherwise.

As will be appreciated by persons skilled in the art, the invention and its embodiments have been described with respect to procedures involving lung tissue, However, certain aspects of the device and method such as the sealing device and component are applicable to other procedures and devices suitable for use elsewhere in the body. These may include but are not limited to kidney, liver, connective tissue, breast, pancreas, spleen, brain, joints, bladder, prostate, mediastinum, muscle, and gastrointestinal tract. Treatment modalities include but are not limited to filling voids in tissue, repairing needle tracts, repairing wounds or deformations, and bulking tissue. Furthermore, the location of implantation of the polymer material may be described as being proximal or distal to a specific site. For example, in the context of a lung biopsy the polymer material may be implanted proximal to a target site such as a cancerous lesion. In this context, proximal is defined as being closer to the pleural cavity than the cancerous lesion such that the polymer material blocks the tract created by the device from the pleural space to the cancerous lesion.

As used herein, the term “coupled” generally means physically coupled or linked and does not exclude the presence of intermediate elements between the coupled items absent specific contrary language.

As used herein, the term “subject” can include a living human subject, cadaver, swine model, canine model, rabbit model, mouse model, or rat model.

As used herein, the term “tissue” can include any tissue within the body. The present invention focuses on the chest wall, pleural space, parietal pleura, visceral pleura, lung parenchyma, bronchioles, alveoli, airways, lung lesions, and lung fissures. However, the device can also relate to different organs including but not limited to kidney, liver, connective tissue, breast, pancreas, spleen, brain, joints, bladder, prostate, mediastinum, muscle, and gastrointestinal tract.

As used herein, the term “target site” is broad and may refer to a cancerous lesion, suspicious lesion, organ, fissure, hemorrhage, arteriovenous malformation, chest wall, pleural space, parietal pleura, visceral pleura, lung parenchyma.

As used herein, the term “polymer” means a molecule, formed by the chemical union of two or more oligomer units. The chemical units are normally linked together by covalent linkages. The two or more combining units in a polymer can be all the same, in which case the polymer is referred to as a homopolymer. They can be also be different and, thus, the polymer will be a combination of the different units. These polymers are referred to as copolymers.

The thermosensitive polymer described herein may also be referred to as “inverse thermosensitive” or “inverse gelling” or “reversibly gelling” which refers to the gelation occurring upon an increase in temperature rather than a decrease in temperature. The temperature at which the polymer transforms to a gel may be referred to as the “transition temperature”.

The term “contrast-enhancing” refers to materials capable of being monitored during injection into a subject by methods of monitoring for detecting such materials for example by CT-Scan, X-Ray, MRI, fluoroscopy, and ultrasound. An example of a contrast-enhancing agent is a radiopaque material. Contrast-enhancing agents including radiopaque materials may be water soluble or water insoluble. Examples of water soluble radiopaque materials include metrizamide, iopamidol, iothalamate sodium, iohexol, iodomide sodium, and meglumine. Examples of water insoluble radiopaque materials include metals and metal oxides such as gold, titanium, silver, stainless steel, nitinol, oxides thereof, aluminum oxide, zirconium oxide, etc.

The term “biocompatible”, as used herein, refers to having the property of being biologically compatible by not producing a toxic, injurious, or immunological response in living tissue.

The terms “poloxamer” and “pluronic” denote a symmetrical block copolymer, consisting of a core of PPG polyoxylethylated to both its terminal hydroxyl groups, i.e conforming to the interchangeable generic formula (PEG)_{X}-(PPG)_{Y}-(PEG)_{X} and (PEO)_{X}-(PPO)_{Y}-(PEO)_{X}. Each poloxamer name ends with an arbitrary code number, which is related to the average numerical values of the respective monomer units denoted by X and Y.

The term “tract” denotes the passage that is created from a device being inserted into tissue. For example, when a coaxial access device is inserted into the lung during a biopsy procedure it creates a tract through the chest wall, pleural space, and lung parenchyma.

The term “bulk” or “bulking”, as used herein, refers to the polymer having sufficient material properties to effectively protrude or expand the surrounding tissue to a size or dimension that is equal or greater to its normal physiologic size or dimension. For example, it may be desirable for the polymer to bulk lung tissue wherein the polymer causes the surrounding lung tissue to expand to a size greater than its native physiologic size wherein the polymer would act as a seal against the passage of fluid such as blood or air.

Examples of thermosensitive polymers described herein include poloxamer 4078, poloxamer 188, Pluronic® F127, Pluronic® F68, poly(N-isopropylacrylamide), poly(methyl vinyl ether), poly(N-vinylcaprolactam); and certain poly(organophosphazenes). See Bull. Korean Chem. Soc. 2002, 23, 549-554. As described herein, the terms Poloxamer 407 and Pluronic® 127 are used interchangeably and refer to the same polymer.

Certain embodiments of the invention are directed to delivery of a hydrogel, sealant, biomaterial, composition, first composition, biosealant, thermosensitive polymers, and temperature sensitive polymers. Thermosensitive polymers are a preferred material for occluding tract that can preferably be delivered to the tissue via a coaxial delivery device such as a percutaneous access device. It was determined that the materials described in this application were chosen to occlude the tissue tract during a transthoracic percutaneous lung biopsy and effectively prevent pneumothorax. The materials developed were created so that they would be easily injected, occlude the tract at the site of delivery, not migrate, and occlude the tract long enough to prevent pneumothorax.

An important aspect of the present invention is the ability to deploy a sealant immediately upon introducing the device into tissue. The present invention reduces the risk of complications associated with accessing the lung parenchyma. The devices and methods are used with sealant compounds which have a suitable density, viscosity, modulus and other material properties to effectively seal the tract to prevent the passage of liquid or gas. Thus, in some embodiments, this disclosure provides a device that can be used to administer, deposit, or deploy (e.g., continuously) a biosealant into a needle tract as a needle is being inserted into a tissue and as the needle is being removed from the tissue, such that air and/or fluids cannot enter the needle tract during or after insertion and/or removal of the needle from the tissue is/are completed.

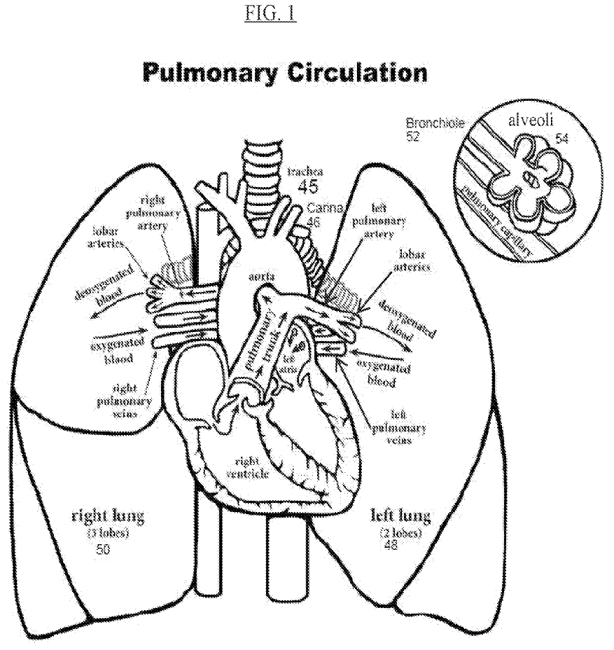

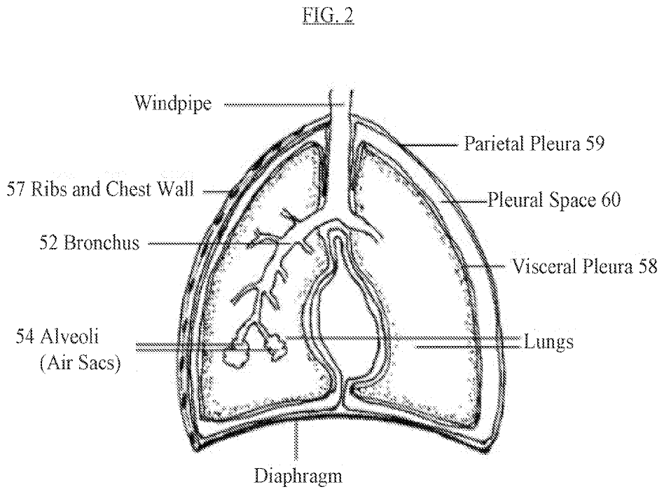

The respiratory system is illustrated in FIG. 1. More specifically the trachea 45 splits at the carina 46 and leads into left lung 48 and right lung 50. The airways, also termed bronchi and bronchioles 52 become smaller as they travel towards the periphery of the lung and eventually end in alveoli 54 which aid in gas exchange. As shown in FIG. 2, the pleural cavity (also called pleural space) 60 is located between the visceral pleura 58 and the parietal pleura 59. The pleural space is a negative pressure area which helps the lungs expand and keeps them inflated within the thoracic cavity which is defined by the ribs and chest wall 57. Both the visceral and parietal pleura are coated with a serous fluid which acts as a lubricant to help them slide and cushion relative to one another during the respiration process. When the pleural space is compromised the lungs can no longer retain their suspended status within the thoracic cavity and they decompress making the respiration process harder on the individual. In most cases, if fluid or gas enters the pleural space an intervention must occur such as a chest tube or surgery.

The pleural space is most commonly compromised when trauma occurs to the chest wall causing a passageway to form from the skin through the lung parenchyma. Lung parenchyma is understood by those of ordinary skill in the art to include those portions of the lung that perform the gas exchange function of the lung, including but not limited to aveoli. This usually results in a spontaneous tension pneumothorax which can place a large outside force on the heart. Iatrogenic pneumothoraces result from transthoracic needle aspiration procedures, EBUS procedures, pleural biopsies, lung biopsies, thoracentesis, tracheostomy, and cardiopulmonary resuscitation. The pleural space can also be filled with fluid such as blood (also referred to as a hemothorax) resulting from blunt trauma, penetrating trauma such as the biopsy methods listed above, nontraumatic or spontaneous neoplasia (primary or metastatic), complications from pulmonary embolisms, torn pleural adhesions, bullous emphysema, tuberculosis, arteriovenous fistulae, thoracic aortic aneurysm, intralobar and extralobar sequestration.

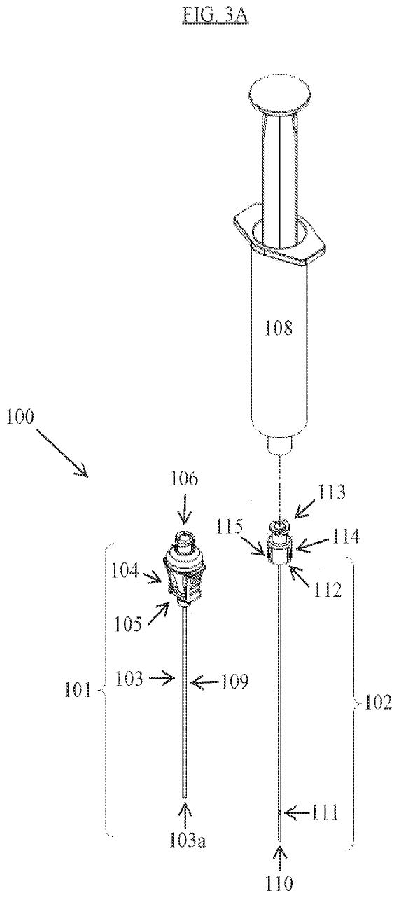

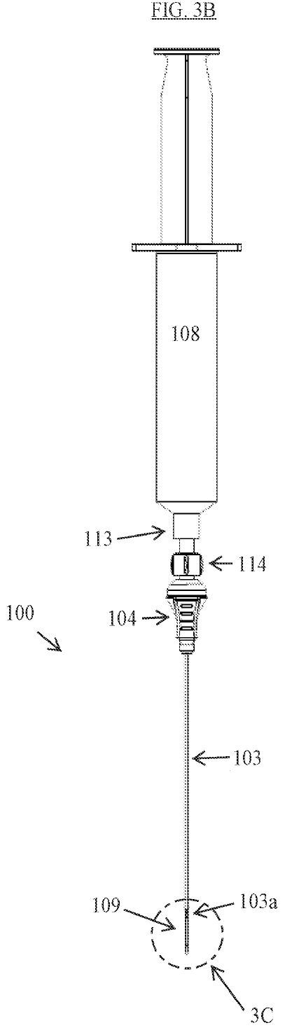

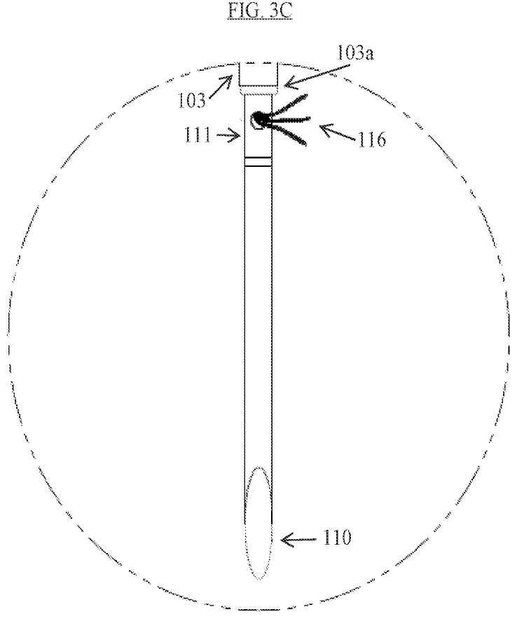

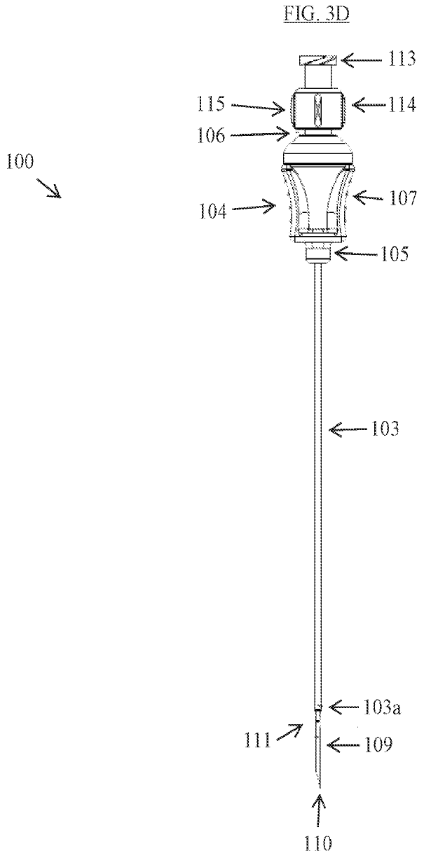

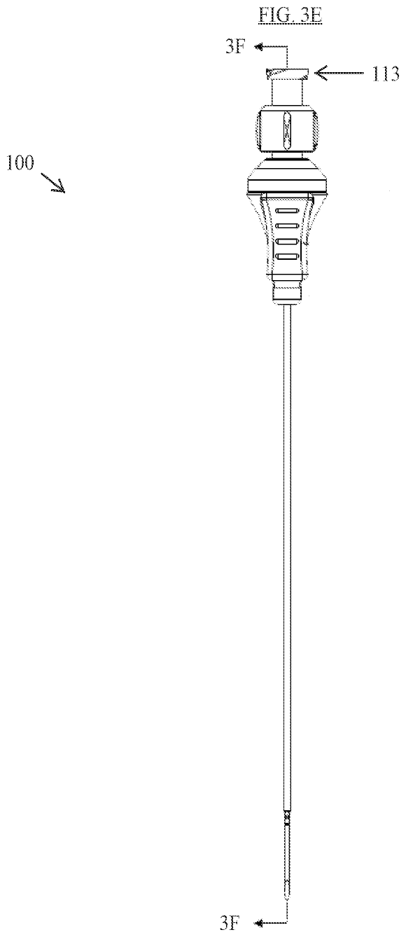

As shown in FIGS. 3A-3C and as referred to above, delivery device 100 consists of a first member 101, second member 102, and optionally a syringe 108. The first member 100 comprises a cannula 103 that connects to hub 104 which comprises a hub distal end 105 and hub proximal end 106. The hub 104 also contains ridges 107 which aid the user with grip, articulation, and insertion of the device into tissue. The cannula 103 has a cannula distal end 103a and is configured to house second member 102 therein. The cannula 103 wall thickness can range from 0.003-0.006 inches (e.g., about 0.003, 0.004, 0.005, or 0.006 inches), and it may have an outer diameter ranging from 14 gauge-24 gauge (e.g., 14, 15, 16, 17, 18, 19, 20, 21, 22, 23 or 24 gauge), or 0.083-0.02225 inches. The cannula 103 may additionally contain graduated markings to indicate the insertion depth of the device to the user, for example the markings may be in 0 cm-30 cm increments, 0 mm to 300 mm increments. Artisans will appreciate that all value between the explicitly stated bounds are contemplated: 0.1 mm, 0.1 cm, 1-20 mm, or 1-7 cm, the graduated marking may be radiopaque so that the user can visualize the depth of the device under imaging modalities such as CT-Scan, X-Ray, MRI, fluoroscopy, and ultrasound. Additionally, the hub 104 contains a central lumen therethrough extending from the hub proximal end 106 to the hub distal end 105 that is connected to and shares a longitudinal axis with cannula 103. Hub proximal end 106 contains male luer-lock system adapted to couple to the female luer-lock system located at hub distal end 112. The hub proximal end 106 may also include an angled orientation relative to the hub distal end 112, for example the hub proximal end 106 may have an angle between 15°-90°. The angled orientation may allow the syringe 108 to be connected to the delivery device 100 during a surgical operation such as a biopsy when a patient is undergoing an image guided therapy such as a CT-guided lung biopsy. For example, the delivery device 100 can be inserted into a patient who is inside a CT gantry and the angled orientation allows the syringe 108 to be connected to the delivery device 100 inside the gantry. Optionally, the hub 104 may contain a septa therein located perpendicular to the longitudinal lumen therethrough to act as a barrier to air or other fluids. The septa may consist of a PTFE, silicon, or combination PTFE-silicon composition. The septa is configured to allow passage of second member 102 therethrough while preventing the passage of air or other fluids thereby providing a tight seal around second member 102 when the second member 102 is inserted through first member 101. The luer lock-type connection is well-known for attaching devices, or units of devices to one another, in which threads on one of the devices and/or units engages opposing threads on the other device and/or unit to provide a leak-free seal, e.g., a rotating lock, and is commonly used in syringe devices (e.g., compatible with industry standard ISO 80369, ISO 594, DIN standard 1707: 1996, and/or EN standard 20594-1:1993). Other suitable methods for connecting the hub proximal end 106 and the hub distal end 112 include but are not limited to screwing, press-fit, snap-fit, molding, and/or one or more adhesives.

The second member 102 comprises a distal tip 110 which has a beveled, tri-faceted, diamond tip, or other sharp tip to facilitate entry into tissue and prevent tissue coring. The second member 102 also comprises a cannula 109 which connects to the hub 114. Located on the cannula 109 is a port 111 which can optionally be one or more ports located on the proximal, central, or distal portion of the cannula 109. However, the preferred embodiment contains a singular port 111 located on a distal end of the cannula 109. The port 111 provides fluid access from the internal lumen of cannula 109 to the outside environment such as a tissue or tract such that a polymer can flow through cannula 109 and out through port 111. The cannula 109 wall thickness can range from 0.003-0.006 inches (e.g., about 0.003, 0.004, 0.005, or 0.006 inches), and it may have an outer diameter ranging from 14 gauge-24 gauge (e.g., 14, 15, 16, 17, 18, 19, 20, 21, 22, 23 or 24 gauge), or 0.083-0.02225 inches. The hub 114 further comprises a hub distal end 112 and a hub proximal end 113. The hub 114 contains a series of grooves 115 which aid the user with grip, articulation, and insertion of the device into tissue. The hub 114 contains a longitudinal lumen therethrough extending from the hub proximal end 113 to the hub distal end 112 wherein the longitudinal lumen axis is aligned with the longitudinal lumen axis of the cannula 109. The hub proximal end 113 contains a male luer-lock connection that is configured to connect to a female luer-lock system of a syringe 108. The syringe 108 is configured to administer a polymer material (11b) that will flow through the longitudinal lumen of the hub 114, through the cannula 109, and out through port 111 into a tissue or tract.

Specific reference is made to FIGS. 3B-3C wherein when the second member 102 is placed within the first member 101 and the hub distal end 112 is coupled to the hub proximal end 106 the cannula distal end 103a is located proximal to the port 111. Optionally, the port 111 may contain a radiopaque identified located at or near the port 111. The radiopaque identifier may help the user more accurate locate the position at which a first composition or polymer 116 is deployed from the device thereby allowing more accurate administration. Therefore, when the second member 102 is placed within the first member 101 the cannula 103 does not prevent the flow of a polymer or first composition 116 to a tissue or tract. The cannula distal end 103a may comprise a taper or a thinner wall thickness to prevent tissue shearing or tearing when the delivery device 100 is inserted into tissue. Furthermore, when the second member 102 is placed within the first member 101 the cannula 103 of the first member 101 completely and circumferentially surround the cannula 109 of the second member 102. Furthermore, the first member 101 and second member 102 are oriented coaxial with one another. The term coaxial is used broadly to encompass a system wherein the central axis of the second member 102, first member 101, and optionally the syringe 108 are located parallel to one another.

FIGS. 3D-3E portray side views of the assembled delivery device 100 wherein the second member 102 is placed within the first member 101 and the hub distal end 112 is coupled to the hub proximal end 106. FIG. 3F shows a cross-sectional view of the delivery device 100. Distal tip 110 may comprise a distinct member that is press-fit and welded, glued, or coupled to the distal end of cannula 109.

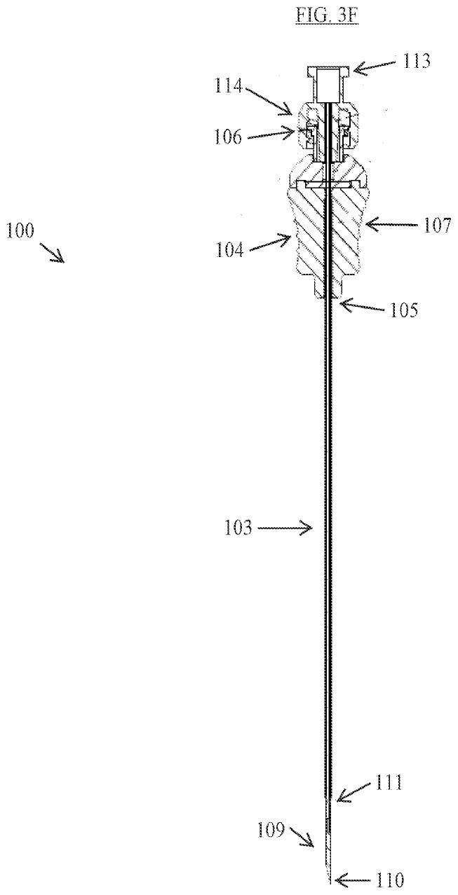

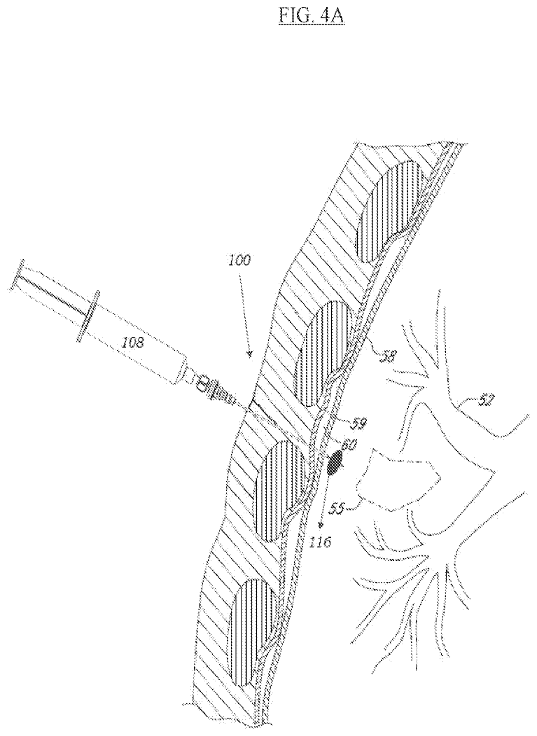

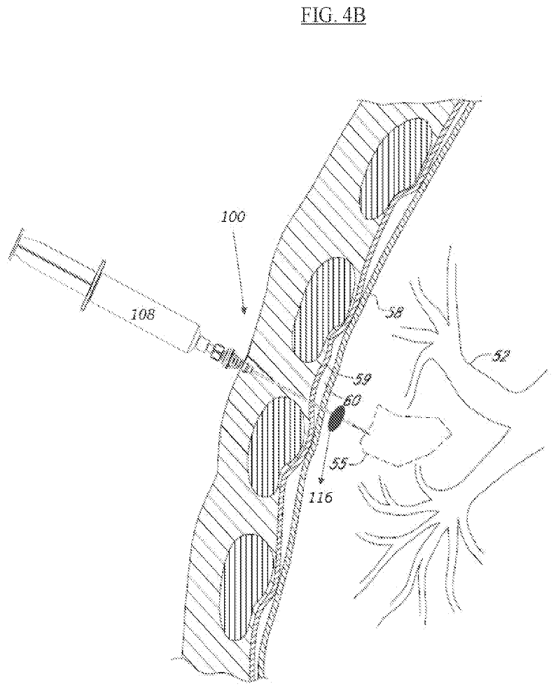

FIGS. 4A-4D relate to a method of using the preferred embodiment of FIGS. 3A-3F. Specifically, FIG. 4A portrays the assembled delivery device 100 wherein the second member 102 is placed within the first member 101 and the hub distal end 112 is coupled to the hub proximal end 106 and syringe 108 is coupled to the delivery device 100. The delivery device is advanced through the chest wall and subsequently the parietal pleura 59, pleural space 60, and the visceral pleura 58 towards a target site 55, preferably a suspicious or cancerous lesion, optionally a bronchi 52. Upon reaching the lung parenchyma located within the visceral pleura a first composition 116, optionally a polymer, optionally a thermosensitive polymer is delivered or administered from the syringe 108 through the delivery device 100 and into the tissue. The first composition 116 comprises a modulus that causes the lung tissue to deform around the first composition 116 thus creating an airtight seal in the tract created by the distal end of the delivery device 100. It will be appreciated that FIG. 4A depicts the first composition 116 in a position that is considered proximal to the target site 55. Additionally, it will be appreciated that the tract is created by the delivery device 100 as it is advanced towards the target site 55. However, the device can also advance along a natural tract such as a fissure or opening containing a natural lumen, optionally a vein, vessel, artery, or nerve conduit. Upon delivery of the first composition 116 air is prevented from being transported to the pleural space 60 from a location distal to the first composition 116 and the first composition provides a circumferential seal around the delivery device 100 such that air does not travel around the outside of the device. Furthermore, the delivery device 100 can be continuously monitored during the operation by imaging modalities such as CT-Scan, X-Ray, MRI, fluoroscopy, and ultrasound.

FIG. 4B illustrates the delivery device 100 being further advanced until the distal end has reached the target site 55. Note that the first composition 116 is still located at the initial site of implantation and does not migrate upon further advancement or movement of the delivery device 100 and acts as a seal against the flow of air while the device moves distal to the first composition 116. FIG. 4C portrays the second member 102 removed from the device while the first member 101 stays in place. The first member 101 acts as a guide or a conduit for a third member 118 which may comprise another device, therapy, treatment, or surgical step to occur. For example, third member 101 may be a biopsy gun (e.g BioPince Full Core Biopsy Instrument (Argon Medical Devices), Quick-Core Biopsy Needle (IZI Medical), Max-Core Biopsy Instrument (Becton Dickinson), Temno Evolution Biopsy Device (Merit Medical)), artisans will appreciate that this list should not be interpreted as limiting but rather an example of the broad amount of biopsy devices that could be utilized and other biopsy manufacturers and modalities such as automatic and fine-needle aspirators have been contemplated and appreciated. Furthermore, first member 101 may allow passage of an ablation device that operates by RF (radio-frequency), microwave, cryoablation, irreversible electroporation.

FIG. 4D shows the device after usage of the third member 118 has been completed and the second member 102 has been re-coupled to the first member 101, optionally the syringe 108 is re-coupled to the delivery device 100. Upon removal of the delivery device 100 a first composition 116 can be delivered or administered to the tissue throughout the tract. For example, the user may administer a first composition 116 from the target site 55 continuously through tract until the delivery device 100 reaches the pleural space 60, preferably the user may decide to selectively administer the first composition 116 proximal to where the first composition 116 was initially administered, preferably the first composition 116 is selectively administered optionally bulked in the lung parenchyma directly adjacent the visceral pleura. Optionally, the first composition 116 can be administered at the visceral pleura, pleural space, bronchi parictal pleura, chest wall, target site or a combination of these structures.

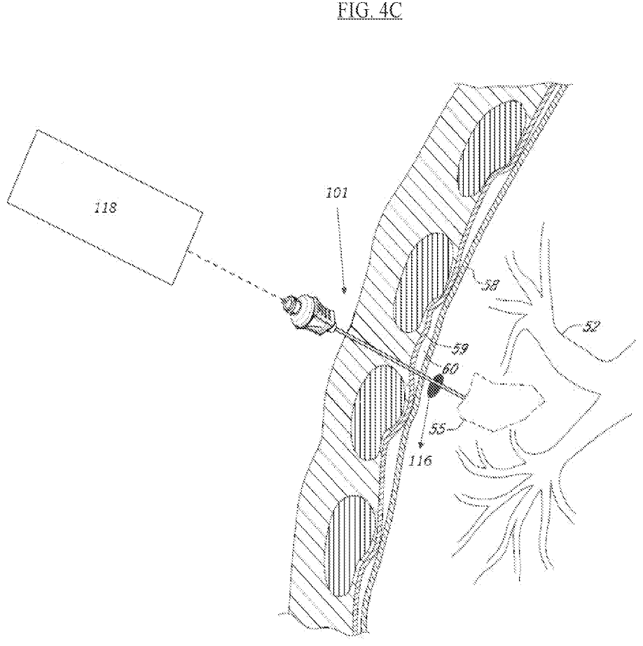

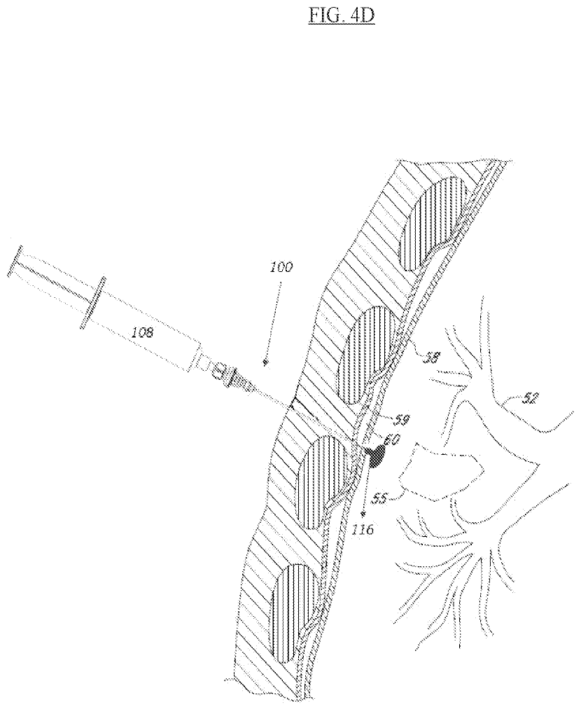

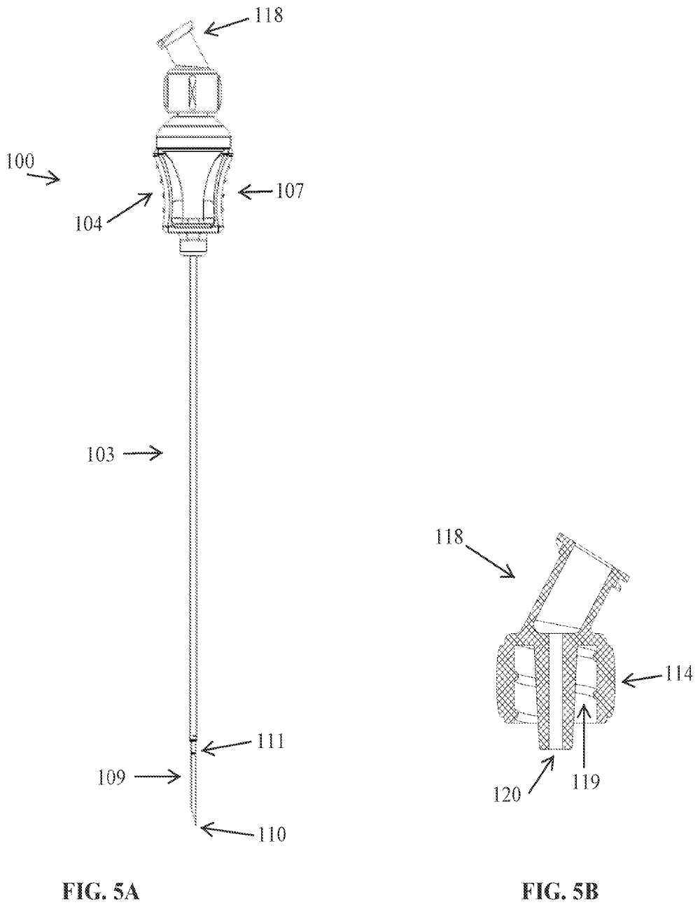

FIG. 5A-5B show an alternative embodiment of the delivery device 100 wherein the second member 102 comprises and angel proximal hub 118. The angled proximal hub 118 allows for a syringe to be fixedly attached to the delivery device 100 at an angle while the patient is undergoing CT-imaging or MRI imaging. The angled proximal hub 118 reduces the total longitudinal length of the device during operation allowing the delivery device 100 optionally with a syringe attached to be inserted into the gantry of a CT-imaging or MRI machine, this proves especially beneficial in patients with a larger body habitus. The angled proximal hub 118 is at an angle relative to the hub 114, additionally the hub 114 has a lumen 120 located therein that fluidly connects to the angled proximal hub 118 which can optionally couple to a syringe containing a first composition. Preferably, the angled proximal hub 118 can have an angle of between 0-90 degrees relative to the hub 114, artisans will appreciate that all angles and ranges within are appreciated (10-20 degrees, 30 degrees, 41-44 degrees, 72, 77, 82, 84, 85.5 degrees, or other appropriate temperature). Furthermore, hub 114 contains threads 119 on the inner wall that can couple to a hub proximal end 113 as shown in FIG. 3A. The threads 119 located on the inner wall of the hub 114 are located outside of the lumen 120. The lumen 120 is defined by a tapered wall thickness wherein the wall thickness closer to the angled proximal hub 118 is greater than the wall thickness towards the distal end of the hub 114. The embodiment shown in FIG. 5B can be either modular wherein it can be coupled to a hub proximal end 113 or it can replace the hub 114 as shown in FIG. 3A wherein the lumen 120 would be fluidly connected to the cannula 109.

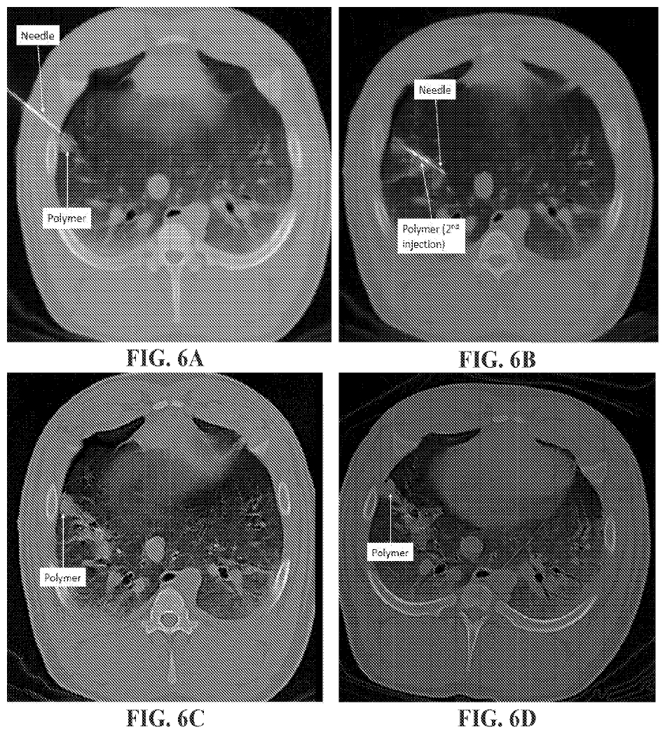

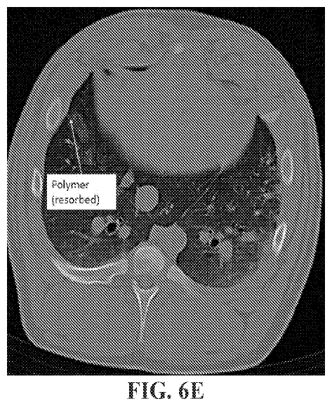

FIGS. 6A-6E show use of the device and polymer to prevent pneumothorax in an in-vivo swine model. Further discussion is provided in the EXAMPLES section below.

Example 1 describes an in vitro test using excised porcine lungs with a polymer material being deployed to act as a seal to prevent pneumothorax. One polymer material consisted of PEGDA (Poly(ethylene glycol) diacrylate) crosslinked with TPVA (thiolated Poly(vinyl) Alcohol), specifically the composition comprised 1.5% PEGDA 3900 w/w crosslinked with 1.9% TPVA w/w. One polymer material consisted of 1.3% chitosan w/w crosslinked with 0.1% genipin. One polymer material consisted of 2% chitosan w/w crosslinked with 11% w/w β-glycerophosphate. One preferred polymer material consisted of 30% Poloxamer 407 w/w diluted in 1×PBS (phosphate buffered saline). In this experiment a series of excised pig lungs were attached to a pressure transducer that inflated the pig lungs. The lungs were then submerged in a water bath to ensure they would reach physiologic internal temperatures. After submersion a coaxial delivery device with a syringe attached was inserted and a polymer material was administered to the tract. The delivery device was then removed while the polymer material was still implanted within the lungs. A successful test was defined as the lungs being submerged and there being no visibility of bubbles emerging from the site of polymer implantation. Results from this experiment are detailed in Table 1. A similar test with a different volume of polymer material injected is detailed in Table 2.

Example 2 used similar materials and testing apparatuses as Example 1 but the polymer materials were intentionally delivered within the lung parenchyma adjacent to the visceral pleura and proximal to the target site. Without being bound to this particular theory, it is believed that a more efficient seal can be created when the polymer material is selected bulked closer to the visceral pleura as compared to a location that is further within the lung parenchyma and therefore a further distance from the visceral pleura and pleural space.

Example 3 utilized 30% w/w Poloxamer 407 diluted in 1×PBS material with 20% w/w iohexol used as a contrast agent and 0.09% w/w FD&C Blue Dye in a live porcine study wherein the polymer material was intentionally administered within the lung parenchyma adjacent to the visceral pleura, the study was performed under fluoroscopic image guidance. Additionally, the thermosensitive polymer was injected in the gel state so that immediately upon administering to the lung tissue it can act as a seal against the passage of air. A successful biopsy of the target site within the lung parenchyma was performed and no pneumothorax occurred. Additionally, the polymer material did not confound the biopsy sample as verified by pathology. The study was a survival study in which after the procedure the endotracheal tube was removed and the pig was able automatically breathe. Another x-ray was taken at a 24-hour timepoint showing the material was still present and did not migrate within the tract. It is significant that these polymer formulations are functional with x-ray contrast agents since this allows users to choose a formulation that is compatible with their existing imaging modalities. Furthermore, the blue dye that was added to the composition aided visualization of the implant location after necropsy, this would potentially have clinical benefit to aid visualization of a lung lesion undergoing resection. For example, the composition can be used as an image-guided tissue marker to aid an open resection of tissue.

Throughout these examples it has been appreciated that different administration techniques may be employed by the user. For example, the user may desire an intermittent delivery technique in which they can administer a portion of the desired dose of polymer material under real-time imaging and assess the results usually within a matter of seconds. Contrarily, the user may administer the full desired dose at one time, the polymer formulation and delivery device allows the user the flexibility to determine the method that best suits their needs.

Example 4 utilized 30% w/w Poloxamer 407 diluted in 1×PBS material without an added contrast agent. The polymer material was injected into a live porcine model wherein the material was administered within a lung parenchymal tract created by a delivery device. The study was performed under CT-guidance which indicated that the formulation without an added contrast agent was visible under CT-imaging.

Other benefits of the preferred 30% w/w Poloxamer 407 diluted in 1×PBS formulation that were observed was that the delivery device did not become occluded or clogged after administering the material and it did not adhere between the tissue and the device reducing the likelihood of tissue tear or shear. Delivery device occlusion is common in the delivery of embolic materials including but not limited to Gelfoam® (Pfizer, New York, NY) and Onyx Liquid Embolic System (Medtronic, Dublin, IE). When occlusion occurs in the delivery device further material cannot be administered until the occlusion is cleared. Example 5 demonstrated that the thermosensitive polymer formulation has low or no adherence to plastic or metal tubing and is not adherent between the plastic or metal tubing and the tissue.

Example 6 demonstrates the injectability force required to administer the polymer material into lung tissue using a 1 cc syringe and a coaxial delivery device. The needle referenced in FIGS. 6A-6B is delivery device 100. However, it is also envisioned that the polymer material can be delivered via a coaxial device, delivery needle, trocar, or standard biopsy kit trocar. Manufacturers of standard biopsy kits include but are not limited to B. D Bard, Merit Medical, IZI Medical, Mermaid Medical.

Example 7 demonstrates the efficacy of the polymer material in preventing pneumothorax on an in-vivo porcine model.

It was also observable that the polymer formulation could be administered in small or large quantities to effectively occlude, bulk, or seal a variety of tract sizes within the lung. For example, the volume of polymer delivered can range from 0.1 cc to 50 cc, artisans will immediately appreciate that all ranges and values between the explicitly stated bounds are contemplated, e.g less than 10 cc or less than 30 cc, 3, 4, 7, 28, 42, 44 cc, from 2-4 cc, at least 2 cc. The polymers when administered to the tract formed a seal directly at the site of injection to block any remaining channels, voids, or areas between the polymer material and the edges of the tract. The polymer materials were formed with adequate mechanical strength of resilience that retarded the flow of blood of fluids throughout the tract, preferably preventing the flow of air into the pleural space.

The general theory of the present invention is that a polymer is needed to occlude the tract of the lung prior to reaching the target site which will provide a continuous seal during the procedure, preventing pneumothorax. Once this theory was appreciated it was also favorable to deploy a polymer material that would provide an immediate seal upon injection into the lung. Therefore, in the context of thermosensitive polymers it was advantageous to administer them in the gel state (at a temperature above the transition temperature) in which the thermosensitive polymer acts as a seal upon immediate injection into the tract. For example, the F127 polymer formulation utilized in the present invention has a gelation temperature of 10° C.; therefore if the ambient temperature is greater than the gelation temperature then it will be injected in a gel state.

### Thermosensitive Hydrogels

Poloxamers, also known as Pluronics®, have unique surfactant abilities and extremely low toxicity and immunogenic responses. Pluronic® polymers are among a small number of surfactants that have been approved by the FDA for direct use in medical applications (Pluromed, Woburn, MA) and (BASF (1990) Pluronic® & Tectronic® Surfactants, BASF Co., Mount Olive N.J). Poloxamers as nonionic surfactants are widely used in industrial applications in which their surfactant properties are useful in detergents, dispersion, stabilization, foaming, and emulsification.

Several Poloxamers show inverse thermosensitivity within physiologic temperature ranges (e.g., poloxamer 188, poloxamer 407, poloxamer 338, poloxamine 1107, and poloxamine 1307). In other words, these polymers are soluble in aqueous solutions at low temperatures but gel at higher temperatures. Poloxamer 407 is a biocompatible polyoxypropylene-polyoxyethylene block copolymer having an average molecular weight of about 12,500 and a polyoxypropylene fraction of about 30%; poloxamer 188 has an average molecular weight of about 8400 and a polyoxypropylene fraction of about 20%; poloxamer 338 has an average molecular weight of about 14,600 and a polyoxypropylene fraction of about 20%; poloxamine 1,107 has an average molecular weight of about 14,000, poloxamine 1307 has an average molecular weight of about 18,000. Polymers of this type are also referred to as reversibly gelling because their viscosity increases and decreases with an increase and decrease in temperature, respectively. Such reversibly gelling systems are useful wherever it is desirable to handle a material in a fluid state, but performance is preferably in a gelled or more viscous state. As noted above, certain poly(ethyleneoxide)/poly(propyleneoxide) block copolymers have these properties; they are available commercially as Pluronic® poloxamers and Tetronic® poloxamines (BASF, Ludwigshafen, Germany) and generically known as poloxamers and poloxamines, respectively. See U.S. Pat. Nos. 4,188,373, 4,478,822 and 4,474,751. Throughout the application terms Pluronic® 127 and Poloxamer 407 represent the same polymer from different manufacturers and are used interchangeably.

Other Poloxamers that are envisioned and considered include but are no limited to: P105, P108, P122, P123, P124, P182, P183, P184, P185, P188, P212, P215, P217, P234, P235, P237, P238, P288, P333, P334, P335, P338, P402, P403, P407. Additionally, their respective Pluronic® names include L35, F38, L42, L43, L44, L62, L63, L64, P65, F68, L72, P75, F77, P84, P85, F87, F88, F98, P103, P104, P105, F108, L122, P123, F127. The formulations listed in the applications herein may contain a singular or multiple combination of the Poloxamer and Pluronic polymers listed above.

The average molecular weights of poloxamers range from about 1,000 to 16,000 daltons. In addition, commercially available poloxamers contain substantial amounts of poly(oxyethylene) homopolymer and poly(oxyethylene)/poly(oxypropylene diblock polymers. The relative amounts of these byproducts increase as the molecular weights of the component blocks of the poloxamer increase. Depending upon the manufacturer, these byproducts may constitute from about 15 to about 50% of the total mass of the polymer.

Thermosensitive polymers may be also used as tissue spacers to prevent thermal, radiation, or cryosurgical damage to surrounding structures. For example, it may be of interest to ablate a lesion that is located in the mediastinum near the heart in which case it may be advantageous to administer a thermosensitive polymer between the heart and the lesion to protect the heart from any collateral damage secondary to treatment on the lesion. Artisans will appreciate that the heart is only one critical structure and serves as an example of the function of the thermosensitive polymer for this application, it can be appreciated that there may be other critical structures around the body including but not limited to the brain, kidney, liver, reproductive organs, etc. . . .

The thermosensitive polymers of the present invention are also suitable delivery vehicles for conventional small-molecule drugs as well as macromolecular drugs such as peptides. Therefore, the thermosensitive polymers may comprise a pharmaceutic or therapeutic agent. The polymers described are capable of solubilizing and releasing pharmaceutic or therapeutic agents. Solubilization will occur as a result of dissolution in the bulk aqueous phase or by incorporation of the solute in micelles created by the hydrophobic domains of the poloxamer. Release of the agent would occur through diffusion or network erosion means.

Preferably the agent that is incorporate into the poloxamer material will be water soluble which lends itself to a homogenous dispersion throughout the thermosensitive composition. The agent incorporated within the poloxamer material can be anesthetic, antimicrobial, antifungal, antiviral, anti-inflammatory, diagnostic, wound killing, and/or cancer killing.

The poloxamer may be combined with a wide array of pharmaceutical agents that may have biological activity, including proteins, polypeptides, polynucleotides, nucleoproteins, polysaccharides, glycoproteins, lipoproteins, and synthetic and biologically engineered analogs thereof.

The poloxamer may be combined with a wide array of therapeutic agents including but not limited to antiinfectives such as antibiotics and antiviral agents; analgesics and analgesic combinations; anorexics; antihelmintics; antiarthritics; antiasthmatic agents; anticonvulsants; antidepressants; antidiuretic agents; antidiarrheals; antihistamines; antiinflammatory agents; antimigraine preparations; antinauseants; antineoplastics; antiparkinsonism drugs; antipruritics; antipsychotics; antipyretics, antispasmodics; anticholinergics; sympathomimetics; xanthine derivatives; cardiovascular preparations including calcium channel blockers and beta-blockers such as pindolol and antiarrhythmics; antihypertensives; diuretics; vasodilators including general coronary, peripheral and cerebral; central nervous system stimulants; cough and cold preparations, including decongestants; hormones such as estradiol and other steroids, including corticosteroids; hypnotics; immunosuppressives; muscle relaxants; parasympatholytics; psychostimulants; sedatives; and tranquilizers; and naturally derived or genetically engineered proteins, polysaccharides, glycoproteins, or lipoproteins.

Additionally, the pharmaceutic agent combined with the poloxamer may be assigned to a certain class of agents; for example, including but not limited to anti-cancer substances, antibiotics, immunosuppressants (e.g., cyclosporine) anti-viral substances, enzyme inhibitors, neurotoxins, opioids, hypnotics, antihistamines, lubricants tranquilizers, anti-convulsants, muscle relaxants and anti-Parkinson substances, anti-spasmodics and muscle contractants, miotics and anticholinergics, anti-glaucoma compounds, anti-parasite and/or anti-protozoal compounds, antihypertensives, analgesics, anti-pyretics and anti-inflammatory agents such as NSAIDs, local anesthetics, ophthalmics, prostaglandins, anti-depressants, anti-psychotic substances, anti-emetics, imaging agents, specific targeting agents, neurotransmitters, proteins, cell response modifiers, and vaccines.

Anti-inflammatory agents may also be added to the thermosensitive polymers of the present invention including but not limited to propionic acid derivatives, acetic acid, fenamic acid derivatives, biphenylcarboxylic acid derivatives, oxicams, including but not limited to aspirin, acetaminophen, ibuprofen, naproxen, benoxaprofen, flurbiprofen, fenbufen, ketoprofen, indoprofen, pirprofen, carporfen, and bucloxic acid and the like.

Anesthetic agents may also be added to the thermosensitive polymers of the present invention including but not limited to bupivacaine, lidocaine, prilocaine, levobupivacaine, ropivacaine, mepivacaine, dibucaine, etidocaine, procaine, amethocaine, benzocaine, tetracaine, alfentanil, fentanyl, buprenorphine, butorphanol, diamorphine, hydromorphone, levorphanol, pethidine, methadone, nalbuphine, oxymorphone, pentazocine.

### Aspects of the Disclosure

In preferred embodiments, this disclosure provides the aspects described below:

A medicament for use in the prevention and/or treatment of pneumothorax in a mammal by administering a first composition comprising a thermosensitive polymer to a group consisting of the chest wall, pleural cavity, internal surface of the visceral pleura, external surface of the parietal pleural, internal surface of the parietal pleura, and/or within the lung parenchyma; wherein the first composition is located proximal to a target site; in preferred embodiments wherein: said thermosensitive polymer is a block polymer, random copolymer, graft polymer or branched copolymer; the first composition has a transition temperature between 5° C. and 40° C.; the first composition comprises 5% to 40% by weight, preferably 20% to 35% by weight of said thermosensitive polymer; the first composition comprises 30% by weight of said thermosensitive polymer; the first composition additionally comprises about 70% by weight of phosphate buffered saline; the thermosensitive polymer is selected from the group consisting of poloxamine 1107, poloxamine 1307, poloxamer 338 and poloxamer 407 (a preferred thermosensitive polymer); the thermosensitive polymer is purified; the target site is selected from the group consisting of a hemorrhage, cancerous tissue, lesion, tumor, and organ; following administration, the distance between the first composition and the pleural cavity is less than the distance between the lesion and the pleural cavity; administration of the thermosensitive polymer creates a tract in a tissue of a mammal, and, the thermosensitive polymer is administered in a gel state thereby temporarily occluding the tract; the first composition comprises a second composition optionally comprising contrast-enhancing agent; in some embodiments, wherein said contrast-enhancing agent is selected from the group consisting of radiopaque materials, paramagnetic materials, heavy atoms, transition metals, lanthanides, actinides, dyes, and radionuclide-containing materials; said pneumothorax is caused by a device inserted into intercostal muscles, visceral pleura, parietal pleura, pleural pleura, or lung parenchyma, preferably by a device inserted into lung parenchyma, optionally wherein the device is selected from the group consisting of an access device, coaxial device, percutaneous delivery device, coaxial delivery device, biopsy device, and ablation device; administration of the thermosensitive polymer creates a tract in a tissue of a mammal, and, the thermosensitive polymer is administered at a proximal location in the tract before the device has reached a target site; said mammal is a human being; the first composition, optionally further comprising the second composition, is injected using a syringe, optionally wherein the syringe has a volume of between about 1 milliliter to about 3 milliliters; and/or, the first composition, optionally further comprising the second composition, is injected using a delivery device, wherein: the delivery device comprises a first member, wherein the first member comprises a cannula and a hub, wherein the hub comprises a hub distal end, a hub proximal end, and ridges; the cannula is connected to the hub distal end; the delivery device further comprising a second member, wherein the second member comprises a cannula, hub, port and sharp closed distal tip configured to pierce through tissue; the hub comprises a hub distal end, a hub proximal end, and grooves; the port is located on the sidewall of the cannula of the second member; the cannula of the second member is configured to connect to the distal end of the hub of the second member; the delivery device has an assembled position wherein the second member is inserted within the first member and the hub distal end of the second member is coupled to the hub proximal end of the first member; in the assembled position the cannula of the second member is concentric with the cannula of the first member; in the assembled position the port is located distal to the distal end of the cannula of the first member; a syringe can be optionally coupled to the hub proximal end of the second member; the syringe comprises the first composition and the first composition is configured to be injected from the syringe through the cannula of the second member and out of the distal port; the first composition comprises a thermosensitive polymer in a gel state; the hub proximal end of the second member is located at an angle relative to the cannula of the first and second members; wherein the hub proximal end is at an angle of between about 15 and 60 degrees relative to the cannula of the first and second members respectively; and/or, the lumen extending through hub proximal end of the second member is located at an angle relative to the cannula of the first and second members.

A system for performing a medical procedure comprising: a delivery device, wherein the delivery device comprises a first member and optionally a second member; wherein the delivery device has an assembled position wherein the second member extends through the first member and a hub distal end of the second member is coupled to a hub proximal end of the first member; wherein the second member further comprises a cannula comprising a port on the sidewall; wherein the port and cannula are fluidly connected to a syringe; wherein the syringe is configured to deliver a thermosensitive gel in a gel state through the port to a target site; wherein the second member further comprises a sharp closed distal tip configured to pierce through tissue; wherein the second member is configured to be de-coupled a removed from the first member during a surgical procedure and the first member is configured to stay in place within a subject; and/or, wherein the first member is configured to allow passage of a different device.

A medicament for use in the prevention and/or treatment of pneumothorax in a mammal by administering a material to a tract within a lung; wherein administration of the thermosensitive polymer causes the lung tissue to bulk; wherein the material is in a gel state and causes the lung tissue to remain bulked; and performing a surgical step in the tract; in some preferred embodiments wherein the surgical step is a lung biopsy or lung ablation; the thermosensitive polymer remains in the gel state; and/or, the thermosensitive polymer comprises poloxamer 407.

A method of administering a first composition at a target site comprising, introducing a delivery device into a tissue; administering a first composition to the tissue; and, advancing the delivery device through the first composition to a target site and performing a surgical step.

Use of a thermosensitive polymer for occluding a tract that is formed due to a percutaneous device, the use comprising; advancing a delivery device to the lung parenchyma; administering a first composition from a side port on the delivery device with the first composition forming a seal around the delivery device; further advancing the delivery device through the first composition to a target site; in some preferred embodiments, wherein: the first composition comprises a thermosensitive polymer; the thermosensitive polymer is selected from the group consisting of poloxamine 1107, poloxamine 1307, poloxamer 338 and poloxamer 407 (in some embodiments, the preferred thermosensitive polymer); and/or, the first composition comprises a liquid and gel state and the first composition is administered in a gel state.

A medical system for the prevention of pneumothorax during percutaneous biopsy, the medical system comprising, a delivery device configured to administer a first composition through a side port; a syringe configured to deliver a first composition in a gel state through the delivery device.

A medical system for the bulking of lung tissue, the medical system comprising, a delivery device configured to administer a first composition through a side port to lung parenchyma, a first composition configured to bulk lung tissue and prevent pneumothorax; wherein in preferred embodiments: the first composition comprises a thermosensitive polymer; the thermosensitive polymer is selected from the group consisting of poloxamine 1107, poloxamine 1307, poloxamer 338 and poloxamer 407 (in some embodiments, the preferred thermosensitive polymer); and/or, the thermosensitive polymer is administered in a gel state.

A method for bulking mammalian tissue comprising the steps of: introducing a first composition to the mammalian tissue that can act as a bulking agent; the first composition comprising a thermosensitive polymer dissolved within a buffer, the thermosensitive polymer comprising about 30% w/w of the composition; wherein in preferred embodiments wherein: the first composition is delivered via a coaxial delivery device; the first composition is delivered through a side port on the coaxial delivery device; the first composition is delivered to the lung parenchyma; the first composition is delivered adjacent to the visceral pleura; and/or, the first composition is delivered in a gel state and does not transform in-situ.

A method of occluding a tract in a mammal comprising the steps of: administering into a tissue of a mammal, at or proximal to a target site, a composition comprising at least one thermosensitive polymer, wherein: administering the thermosensitive polymer creates a tract in the tissue, and, the thermosensitive polymer is in a gel state and thereby immediately occluding the tract; wherein in preferred embodiments: the method further comprises a surgical procedure; the thermosensitive polymer is a poloxamer or poloxamine; at least one thermosensitive polymer is selected from the group consisting of poloxamer 407, poloxamer 338, poloxamer 118, poloxamine 1107 or poloxamine 1307; the at least one thermosensitive polymer is poloxamer 407; the shape of the composition in the tract is selected from the group consisting of a substantially circular, substantially elliptical right cylinder, a substantially circular cylinder, substantially elliptical oblique cylinder, a substantially circular truncated cone, and a substantially elliptical right truncated cone; said composition comprises at least one thermosensitive polymer selected from the group consisting of a block copolymer, random copolymer, graft polymer, branched copolymer, or a combination thereof; said at least one thermosensitive polymer is poloxamer 407; said composition has a transition temperature of between about 5° C. and 15° C.; said tissue is selected from the group consisting of visceral pleura, parietal pleura, pleural cavity, lung parenchyma, kidney, and liver; the surgical procedure is selected from the group consisting of a lung biopsy, a percutaneous lung biopsy, a transthoracic lung biopsy, a lung ablation, a percutaneous lung ablation, and transthoracic lung ablation; the composition in the tract reduces complications selected from the group consisting of bleeding, hemorrhage, pneumothorax, pulmonary air embolism, and tumor tract seeding; said target site is selected from the group consisting of a hemorrhage, cancerous tissue, tumor, organ, spleen, thyroid, thymus, lymphoid tissue, adrenal gland, or dural sac; said composition occludes said tract for greater than 24 hours; said composition occludes said tract for at least 4 hours; said composition is introduced to said tissue via a device selected from the group consisting of a percutaneous access device, coaxial access device, needle, and catheter, wherein in some embodiments: said coaxial access device comprises a first member, wherein the first member comprises a cannula and a hub, wherein the hub comprises a hub distal end, a hub proximal end, and ridges; the cannula is connected to the hub distal end; the delivery device further comprising a second member, wherein the second member comprises a cannula, hub, and port; the hub comprises a hub distal end, a hub proximal end, and grooves; the port is located on the sidewall of the cannula of the second member; the cannula of the second member is configured to connect to the distal end of the hub of the second member; the delivery device has an assembled position wherein the second member is inserted within the first member and the hub distal end of the second member is coupled to the hub proximal end of the first member; in the assembled position the cannula of the second member is concentric with the cannula of the first member; in the assembled position the port is located distal to the distal end of the cannula of the first member; a syringe can be optionally coupled to the hub proximal end of the second member; wherein the syringe comprises the first composition and the first composition is configured to be injected from the syringe through the cannula of the second member and out of the distal port; and/or, the proximal hub of the second member is located at an angle relative to the cannula of the first member and second member; said composition is administered to said tissue using a syringe; said composition is administered to said tissue using a syringe with a volume of between about 0.1 milliliters to 10 milliliters; and said volume is between 1 milliliter to 3 milliliters.

A method for preventing pneumothorax comprising inserting a delivery device into lung parenchyma; and, administering a first composition in a sufficient volume to bulk the lung parenchyma; wherein in preferred embodiments: the delivery device is a coaxial delivery device; the first composition is a thermosensitive polymer that can be selected from the group consisting of poloxamer 407, poloxamer 338, poloxamer 118, poloxamine 1107 or poloxamine 1307; the thermosensitive polymer is dissolved in a carrier resulting in an injectable composition; the carrier comprises a phosphate buffer; the thermosensitive polymer comprises poloxamer 407; poloxamer 407 comprises about 30% w/w of the composition; a syringe containing the first composition is fixedly attached to the coaxial delivery device, wherein in preferred embodiments: the coaxial delivery device comprises a first member having a cannula, a port, and closed sharp distal tip configured to pierce through tissue; the first member has a proximal hub that has an axis therethrough that is located at an off-angle relative to the cannula; the coaxial delivery device comprises a second member comprising a cannula that allows passage therethrough of the first member; the coaxial delivery device comprises an assembled position wherein the first member is housed within and coupled to the second member; after administering a first composition the delivery device travels through the first composition to a target site; the closed sharp distal tip is located distal to the first composition when the first composition is administered; the first composition is configured to form a seal around the delivery device when administered; the first composition is injected in a gel state and does not transform in-situ; and/or, the first composition is injected in a gel state and does not transform in-situ.

A device comprising: a cannula ending in a closed sharp distal tip configured to pierce through tissue; a proximal hub comprising a distal end and proximal end; the distal end of the proximal hub configured to couple to the proximal end of the cannula; and, the hub proximal end having an off angle relative to the cannula; wherein in some embodiments: the cannula contains a side port; the side port is located on the distal end of the device; the side port is fluidly connected to the proximal hub; and/or, the hub proximal end is configured to couple to a syringe.

A method for reducing or preventing complications during lung biopsy comprising administering to the lung parenchyma a first composition comprising a thermosensitive polymer with a delivery device; wherein in preferred embodiments: the thermosensitive polymer is selected from the group consisting of poloxamer 407, poloxamer 338, poloxamer 118, poloxamine 1107 or poloxamine 1307 (in preferred embodiments wherein the thermosensitive polymer comprises poloxamer 407); the thermosensitive polymer is dissolved in a buffer that creates an injectable material; the buffer is a phosphate buffer; the thermosensitive polymer comprises 30% w/w of poloxamer 407, wherein the composition optionally comprises at least 0.05% w/w of a dye (preferably wherein the dye comprises FD&C Blue); the first composition is administered in a gel state and does not transform in-situ; the thermosensitive polymer is administered adjacent to the visceral pleura; the delivery device is inserted towards a target site, wherein the delivery device is configured to deliver the first composition at a location proximal to the target site before the target site has been reached; the delivery device forms a tract, upon administering the first composition the tract becomes occluded; the delivery device has a closed sharp distal end configured to pierce through tissue, a cannula, a hub, and a port located on the sidewall of the cannula; the hub comprises a hub proximal end that can couple to a syringe wherein the syringe can deliver the first composition from the syringe through the cannula, through the port, and to the tract; the syringe comprises a volume between about 1 ml to 3 ml; the first composition is injected in a gel state and the first composition does not transform in-situ; the first composition forms around the entire outer cannula of the delivery device thereby forming a seal; the closed sharp distal end is located distal to the first composition when the first composition is administered; after the first composition is administered the delivery device is advanced to the target site and the first composition stays in place; the first composition stays within the tract for at least 4 hours; and/or, after administering the first composition the first composition does not occlude the cannula of the delivery device.

A medicament for preventing complications during percutaneous transthoracic procedures comprising: advancing a delivery device to lung parenchyma wherein the advancement of the delivery device creates a void in the lung parenchyma; administering a first composition comprising a thermosensitive polymer through the delivery device and filling the void; wherein in preferred embodiments: the thermosensitive polymer is selected from the group consisting of poloxamer 407, poloxamer 338, poloxamer 118, poloxamine 1107 or poloxamine 1307; the thermosensitive polymer comprises poloxamer 407; the first composition comprises between about 10% to 40% w/w of the thermosensitive polymer; the thermosensitive polymer is delivered in a gel state and does not transform in-situ; the first composition is delivered through a side port located on the delivery device; the side port is located proximal to a sharp closed distal end of the delivery device; the first composition fills the void for at least 4 hours; and/or, the first composition is biodegradable.

A medicament for use in the prevention and/or treatment of pneumothorax in a mammal by administering a first composition comprising a thermosensitive polymer to a group consisting of the pleural cavity, internal surface of the visceral pleura, external surface of the parietal pleural, internal surface of the parietal pleura, and/or within the lung parenchyma; wherein the first composition is located proximal to a target site; preferably in some embodiments wherein said thermosensitive polymer is a block polymer, random copolymer, graft polymer or branched copolymer; preferably in some embodiments wherein the first composition has a transition temperature between 5° C. and 40° C.; preferably in some embodiments wherein the first composition comprises 5% to 40% by weight, preferably 20% to 35% by weight of said thermosensitive polymer; preferably in some embodiments wherein the first composition comprises 30% by weight of said thermosensitive polymer; preferably in some embodiments wherein the first composition additionally comprises about 70% by weight of phosphate buffered saline; preferably in some embodiments wherein the thermosensitive polymer is selected from the group consisting of poloxamine 1107, poloxamine 1307, poloxamer 338 and especially preferred poloxamer 407; preferably in some embodiments wherein the thermosensitive polymer is purified; preferably in some embodiments wherein the target site is selected from the group consisting of a hemorrhage, cancerous tissue, lesion, tumor, and organ; preferably in some embodiments wherein following administration, the distance between the first composition and the pleural cavity is less than the distance between the target site and the pleural cavity; preferably in some embodiments wherein administration of the thermosensitive polymer creates a tract in a tissue of a mammal, and, the thermosensitive polymer is administered in a gel state thereby temporarily occluding the tract; preferably in some embodiments where the first composition comprises a second composition optionally comprising a contrast-enhancing agent; preferably in some embodiments where said contrast-enhancing agent is selected from the group consisting of radiopaque materials, paramagnetic materials, heavy atoms, transition metals, lanthanides, actinides, dyes, and radionuclide-containing materials; preferably in some embodiments wherein said pneumothorax is caused by a device inserted into intercostal muscles, visceral pleura, parietal pleura, pleural pleura, or lung parenchyma, preferably by a device inserted into lung parenchyma, optionally wherein the device is selected from the group consisting of an access device, coaxial device, percutaneous delivery device, coaxial delivery device, biopsy device, and ablation device; preferably in some embodiments wherein administration of the thermosensitive polymer creates a tract in a tissue of a mammal, and, the thermosensitive polymer is administered at a proximal location in the tract before the device has reached a target site; preferably in some embodiments wherein said mammal is a human; preferably in some embodiments wherein the first composition, optionally further comprising the second composition, is injected using a syringe, optionally wherein the syringe has a volume of between about 1 milliliter to about 3 milliliters; preferably in some embodiments wherein the first composition, optionally further comprising the second composition, is injected using a delivery device, wherein the delivery device comprises a first member, wherein the first member comprises a cannula and a hub, wherein the hub comprises a hub distal end, a hub proximal end, and ridges, wherein the cannula is connected to the hub distal end, the delivery device further comprising a second member, wherein the second member comprises a cannula, hub, port and sharp closed distal tip configured to pierce through tissue, wherein the hub comprises a hub distal end, a hub proximal end, and grooves, wherein the port is located on the sidewall of the cannula of the second member, wherein the cannula of the second member is configured to connect to the distal end of the hub of the second member, wherein the delivery device has an assembled position wherein the second member is inserted within the first member and the hub distal end of the second member is coupled to the hub proximal end of the first member, wherein in the assembled position the cannula of the second member is concentric with the cannula of the first member, wherein in the assembled position the port is located distal to the distal end of the cannula of the first member, wherein a syringe can be optionally coupled to the hub proximal end of the second member, wherein the syringe comprises the first composition and the first composition is configured to be injected from the syringe through the cannula of the second member and out of the distal port, and wherein the first composition comprises a thermosensitive polymer in a gel state, preferably in some embodiments wherein the hub proximal end of the second member is located at an angle relative to the cannula of the first and second members, and/or preferably in some embodiments wherein the lumen extending through hub proximal end of the second member is located at an angle relative to the cannula of the first and second members.

A system for performing a medical procedure comprising: a delivery device, wherein the delivery device comprises a first member and optionally a second member, wherein the delivery device has an assembled position wherein the second member extends through the first member and a hub distal end of the second member is coupled to a hub proximal end of the first member, wherein the second member further comprises a cannula comprising a port on the sidewall, wherein the port and cannula are fluidly connected to a syringe, wherein the syringe is configured to deliver a thermosensitive gel in a gel state through the port to a target site, wherein the second member further comprises a sharp closed distal tip configured to pierce through tissue, wherein the second member is configured to be de-coupled a removed from the first member during a surgical procedure and the first member is configured to stay in place within a subject, and/or wherein the first member is configured to allow passage of a different device.

A medicament for use in the prevention and/or treatment of pneumothorax in a mammal by administering a material to a tract within a lung, wherein administration of the thermosensitive polymer causes the lung tissue to bulk, wherein the material is in a gel state and causes the lung tissue to remain bulked, and performing a surgical step in the tract; preferably in some embodiments wherein the surgical step is a lung biopsy; preferably in some embodiments wherein the thermosensitive polymer remains in the gel state; preferably in some embodiments wherein the thermosensitive polymer comprises poloxamer 407.

A method of administering a first composition at a target site comprising, introducing a delivery device into a tissue, administering a first composition to the tissue, advancing the delivery device through the first composition to a target site and performing a surgical step.

Use of a thermosensitive polymer for occluding a tract that is formed due to a percutaneous device, the use comprising, advancing a delivery device to the lung parenchyma, administering a first composition from a side port on the delivery device with the first composition forming a seal around the delivery device, and further advancing the delivery device through the first composition to a target site; preferably in some embodiments wherein the first composition comprises a thermosensitive polymer; preferably in some embodiments wherein the thermosensitive polymer is selected from the group consisting of poloxamine 1107, poloxamine 1307, poloxamer 338 and poloxamer 407; preferably in some embodiments wherein the thermosensitive polymer is poloxamer 407; preferably in some embodiments wherein the first composition comprises a liquid and gel state and the first composition is administered in a gel state.

A medical system for the prevention of pneumothorax during percutaneous biopsy, the medical system comprising, a delivery device configured to administer a first composition through a side port; a syringe configured to deliver a first composition in a gel state through the delivery device; and/or a medical system for the bulking of lung tissue, the medical system comprising, a delivery device configured to administer a first composition through a side port to lung parenchyma, a first composition configured to bulk lung tissue and prevent pneumothorax; preferably in some embodiments wherein the first composition comprises a thermosensitive polymer; preferably in some embodiments wherein the thermosensitive polymer is selected from the group consisting of poloxamine 1107, poloxamine 1307, poloxamer 338 and poloxamer 407; preferably in some embodiments wherein the thermosensitive polymer is poloxamer 407; and/or, preferably in some embodiments wherein the thermosensitive polymer is administered in a gel state.

A method for bulking mammalian tissue comprising the steps of: introducing a first composition to the mammalian tissue that can act as a bulking agent; the first composition comprising a thermosensitive polymer dissolved within a buffer, the thermosensitive polymer comprising about 30% w/w of the composition; preferably in some embodiments wherein the first composition is delivered via a coaxial delivery device; preferably in some embodiments wherein the first composition is delivered through a side port on the coaxial delivery device; preferably in some embodiments wherein the first composition is delivered to the lung parenchyma; preferably in some embodiments wherein the first composition is delivered adjacent to the visceral pleura; and/or, preferably in some embodiments wherein the first composition is delivered in a gel state and does not transform in-situ.

A method of occluding a tract in a mammal comprising the steps of: administering into a tissue of a mammal, at or proximal to a target site, a composition comprising at least one thermosensitive polymer, wherein: administering the thermosensitive polymer creates a tract in the tissue, and, the thermosensitive polymer is in a gel state and thereby immediately occluding the tract; preferably in some embodiments further comprising a surgical procedure; preferably in some embodiments wherein the thermosensitive polymer is a poloxamer or poloxamine; preferably in some embodiments wherein the at least one thermosensitive polymer is selected from the group consisting of poloxamer 407, poloxamer 338, poloxamer 118, poloxamine 1107 or poloxamine 1307; preferably in some embodiments wherein the at least one thermosensitive polymer is poloxamer 407; preferably in some embodiments wherein the shape of the composition in the tract is selected from the group consisting of a substantially circular, substantially elliptical right cylinder, a substantially circular cylinder, substantially elliptical oblique cylinder, a substantially circular truncated cone, and a substantially elliptical right truncated cone; preferably in some embodiments wherein said composition comprises at least one thermosensitive polymer selected from the group consisting of a block copolymer, random copolymer, graft polymer, branched copolymer, or a combination thereof; preferably in some embodiments wherein said at least one thermosensitive polymer is poloxamer 407; preferably in some embodiments wherein said composition has a transition temperature of between about 5° C. and 15° C.; preferably in some embodiments wherein said tissue is selected from the group consisting of visceral pleura, parietal pleura, pleural cavity, lung parenchyma, kidney, and liver; preferably in some embodiments wherein the surgical procedure is selected from the group consisting of a lung biopsy, a percutaneous lung biopsy, a transthoracic lung biopsy, a lung ablation, a percutaneous lung ablation, and transthoracic lung; preferably in some embodiments wherein the composition in the tract reduces complications selected from the group consisting of bleeding, hemorrhage, pneumothorax, pulmonary air embolism, and tumor tract seeding; preferably in some embodiments wherein said target site is selected from the group consisting of a hemorrhage, cancerous tissue, tumor, and organ; preferably in some embodiments wherein said composition occludes said tract for greater than 24 hours; preferably in some embodiments wherein said composition occludes said tract for at least 4 hours; preferably in some embodiments wherein said composition is introduced to said tissue via a device selected from the group consisting of a percutaneous access device, coaxial access device, needle, and catheter; preferably in some embodiments wherein said coaxial access device comprises a first member, wherein the first member comprises a cannula and a hub, wherein the hub comprises a hub distal end, a hub proximal end, and ridges, wherein the cannula is connected to the hub distal end, the delivery device further comprising a second member, wherein the second member comprises a cannula, hub, and port, wherein the hub comprises a hub distal end, a hub proximal end, and grooves, wherein the port is located on the sidewall of the cannula of the second member, wherein the cannula of the second member is configured to connect to the distal end of the hub of the second member, wherein the delivery device has an assembled position wherein the second member is inserted within the first member and the hub distal end of the second member is coupled to the hub proximal end of the first member, wherein in the assembled position the cannula of the second member is concentric with the cannula of the first member, wherein in the assembled position the port is located distal to the distal end of the cannula of the first member, wherein a syringe can be optionally coupled to the hub proximal end of the second member, and wherein the syringe comprises the first composition and the first composition is configured to be injected from the syringe through the cannula of the second member and out of the distal port; preferably in some embodiments wherein the proximal hub of the second member is located at an angle relative to the cannula of the first member and second member; preferably in some embodiments wherein said composition is administered to said tissue using a syringe; preferably in some embodiments wherein said composition is administered to said tissue using a syringe with a volume of between about 0.1 milliliters to 10 milliliters; preferably in some embodiments wherein said volume is between 1 milliliter to 3 milliliters.

A method for preventing pneumothorax comprising: inserting a delivery device into lung parenchyma; and, administering a first composition in a sufficient volume to bulk the lung parenchyma; preferably in some embodiments wherein the delivery device is a coaxial delivery device; preferably in some embodiments wherein the first composition is a thermosensitive polymer; preferably in some embodiments wherein the thermosensitive polymer is selected from the group consisting of poloxamer 407, poloxamer 338, poloxamer 118, poloxamine 1107 or poloxamine 1307; preferably in some embodiments wherein the thermosensitive polymer is dissolved in a carrier resulting in an injectable composition; preferably in some embodiments wherein the carrier comprises a phosphate buffer; preferably in some embodiments wherein the thermosensitive polymer comprises poloxamer 407; preferably in some embodiments wherein poloxamer 407 comprises about 30% w/w of the composition; preferably in some embodiments wherein a syringe containing the first composition is fixedly attached to the coaxial delivery device; preferably in some embodiments wherein the coaxial delivery device comprises a first member having a cannula, a port, and closed sharp distal tip configured to pierce through tissue; preferably in some embodiments wherein the first member has a proximal hub that has a axis therethrough that is located at an off-angle relative to the cannula; preferably in some embodiments wherein the coaxial delivery device comprises a second member comprising a cannula that allows passage therethrough of the first member; preferably in some embodiments wherein the coaxial delivery device comprises an assembled position wherein the first member is housed within and coupled to the second member; preferably in some embodiments wherein after administering a first composition the delivery device travels through the first composition to a target site; preferably in some embodiments wherein the closed sharp distal tip is located distal to the first composition when the first composition is administered; preferably in some embodiments wherein the first composition is configured to form a seal around the delivery device when administered; preferably in some embodiments wherein the first composition is injected in a gel state and does not transform in-situ; and/or, preferably in some embodiments wherein the first composition is injected in a gel state and does not transform in-situ.

A device comprising: a cannula ending in a closed sharp distal tip configured to pierce through tissue, a proximal hub comprising a distal end and proximal end, the distal end of the proximal hub configured to couple to the proximal end of the cannula, and the hub proximal end having an off angle relative to the cannula; preferably in some embodiments wherein the cannula contains a side port; preferably in some embodiments wherein the side port is located on the distal end of the device; preferably in some embodiments wherein the side port is fluidly connected to the proximal hub; and/or, preferably in some embodiments wherein the hub proximal end is configured to couple to a syringe.

A method for reducing or preventing complications during lung biopsy comprising administering to the lung parenchyma a first composition comprising a thermosensitive polymer with a delivery device; preferably in some embodiments wherein the thermosensitive polymer is selected from the group consisting of poloxamer 407, poloxamer 338, poloxamer 118, poloxamine 1107 or poloxamine 1307; preferably in some embodiments wherein the thermosensitive polymer comprises poloxamer 407; preferably in some embodiments wherein the thermosensitive polymer is dissolved in a buffer that creates an injectable material; preferably in some embodiments wherein the buffer is a phosphate buffer; preferably in some embodiments wherein the thermosensitive polymer comprises 30% w/w of poloxamer 407, wherein the composition optionally comprises at least 0.05% w/w of a dye; preferably in some embodiments wherein the dye comprises FD&C Blue; preferably in some embodiments wherein the first composition is administered in a gel state and does not transform in-situ; preferably in some embodiments wherein the thermosensitive polymer is administered adjacent to the visceral pleura; preferably in some embodiments wherein the delivery device is inserted towards a target site, wherein the delivery device is configured to deliver the first composition at a location proximal to the target site before the target site has been reached; preferably in some embodiments wherein the delivery device forms a tract, upon administering the first composition the tract becomes occluded; preferably in some embodiments wherein the delivery device has a closed sharp distal end configured to pierce through tissue, a cannula, a hub, and a port located on the sidewall of the cannula; preferably in some embodiments wherein the hub comprises a hub proximal end that can couple to a syringe wherein the syringe can deliver the first composition from the syringe through the cannula, through the port, and to the tract; preferably in some embodiments wherein the syringe comprises a volume between about 1 ml to 3 ml; preferably in some embodiments wherein the first composition is injected in a gel state and the first composition does not transform in-situ; preferably in some embodiments wherein the first composition forms around the entire outer cannula of the delivery device thereby forming a seal; preferably in some embodiments wherein the closed sharp distal end is located distal to the first composition when the first composition is administered; preferably in some embodiments wherein after the first composition is administered the delivery device is advanced to the target site and the first composition stays in place; preferably in some embodiments wherein the first composition stays within the tract for at least 4 hours; and/or preferably in some embodiments wherein after administering the first composition the first composition does not occlude the cannula of the delivery device.

A medicament for preventing complications during percutaneous transthoracic procedures comprising: advancing a delivery device to lung parenchyma wherein the advancement of the delivery device creates a void in the lung parenchyma; administering a first composition comprising a thermosensitive polymer through the delivery device and filling the void; preferably in some embodiments wherein the thermosensitive polymer is selected from the group consisting of poloxamer 407, poloxamer 338, poloxamer 118, poloxamine 1107 or poloxamine 1307; preferably in some embodiments wherein the thermosensitive polymer comprises poloxamer 407; preferably in some embodiments wherein the first composition comprises between about 10% to 40% w/w of the thermosensitive polymer; preferably in some embodiments wherein the thermosensitive polymer is delivered in a gel state and does not transform in-situ; preferably in some embodiments wherein the first composition is delivered through a side port located on the delivery device; preferably in some embodiments wherein the side port is located proximal to a sharp closed distal end of the delivery device; preferably in some embodiments wherein the first composition fills the void for at least 4 hours; preferably in some embodiments wherein the first composition is biodegradable.

A medicament for use in the prevention and/or treatment of pneumothorax in a mammal by administering a first composition comprising a thermosensitive polymer to a group consisting of the pleural cavity, internal surface of the visceral pleura, external surface of the parietal pleural, internal surface of the parietal pleura, and/or within the lung parenchyma; wherein the first composition is located proximal to a target site; preferably in some embodiments wherein said thermosensitive polymer is a block polymer, random copolymer, graft polymer or branched copolymer; preferably in some embodiments wherein the first composition has a transition temperature between 5° C. and 40° C.; preferably in some embodiments wherein the first composition comprises 5% to 40% by weight, preferably 20% to 35% by weight of said thermosensitive polymer; preferably in some embodiments wherein the first composition comprises 30% by weight of said thermosensitive polymer; preferably in some embodiments wherein the thermosensitive polymer is purified; preferably in some embodiments wherein the target site is selected from the group consisting of a hemorrhage, cancerous tissue, lesion, tumor, and organ; preferably in some embodiments wherein following administration, the distance between the first composition and the pleural cavity is less than the distance between the target site and the pleural cavity; preferably in some embodiments wherein, following administration, the distance between the first composition and the pleural cavity is less than 2 centimeters; preferably in some embodiments wherein administration of the thermosensitive polymer creates a tract in a tissue of a mammal, and, the thermosensitive polymer is administered in a gel state thereby temporarily occluding the tract; preferably in some embodiments wherein the thermosensitive polymer is administered via a coaxial device; preferably in some embodiments wherein the thermosensitive polymer degrades between 2 hours and 2 weeks; and/or, preferably in some embodiments wherein the thermosensitive polymer is fully degraded within 24 hours.

Other embodiments of the same are also contemplated herein, as would be understood by those of ordinary skill in the art.

### EXAMPLES

The invention now being generally described, it will be more readily understood by reference to the following examples, which are included merely for purposes of illustration of certain aspects and embodiments of the present invention and are not intended to limit the invention.

### Example 1: In-Vitro Evaluation of Polymer Formulations

A pair of excised pig lungs were attached to a ventilation machine which inflated the lungs to a constant pressure of 10 mmHg (slightly above the physiologic normal pleural pressure of −4 mmHg). The lungs were submerged in a water bath that was held at 37° C. to mimic physiologic body temperatures. A percutaneous coaxial delivery device as shown in FIGS. 3A-3F was used to deliver an array of different polymer formulations. The delivery device was inserted 0.5 cm deep into the lung tissue and specified volume of polymer material was injected with a 1 mL volume polycarbonate syringe (BD Disposable Syringe, Fisher Scientific). After injection the delivery device was removed and the lungs were observed for 5 minutes. The test was considered a success if no bubbles were visualized for 5 minutes. A summary of the results wherein 0.25 mL of material was injected can be seen in Table 1 below. A summary of the results wherein 1 mL of material was injected can be seen in Table 2 below.

TABLE 1

| Polymer Formulation (w/w) | Sealing Success Rate (%) | |
|---|---|---|
| T-Chitosan - 2.0% | 7/11 | (64%) |
| PEGDa 700 - 5.0% | | |
| T-Chitosan - 2.0% | 6/9 | (67%) |
| PEGDa 700 - 10.0% | | |
| T-Chitosan - 2.0% | 10/11 | (91%) |
| PEGDa 700 - 20.0% | | |
| TPVA - 1.9% | 12/13 | (92%) |
| PEGDa 3400 - 1.5% | | |
| TPVA - 3.8% | 11/13 | (85%) |
| PEGDa 3400 - 0.7% | | |
| TPVA - 3.8% | 5/5 | (100%) |
| PEGDa 3400 - 1% | | |

TABLE 2

| Polymer Formulation (w/w) | Sealing Success Rate (%) | |
|---|---|---|
| Poloxamer 407 - 30% in | 10/10 | (100%) |
| 1x PBS | | |
| Pluronic ® F68 - 55% in | 3/6 | (50%) |
| 1x PBS | | |
| Chitosan - 0.7% | 4/5 | (80%) |
| β-Glycerophosphate - 11.2% | | |
| Chitosan - 2% | 9/10 | (90%)_{—} |
| β-Glycerophosphate - 11% | | |
| Chitosan - 2% | 4/5 | (80%) |
| β-Glycerophosphate - 11% | | |
| Gelatin - 1% | | |
| TPVA - 1.9% | 10/10 | (100%) |
| PEGDa 3400 - 1.5% | | |
| Chitosan - 1.3% | 10/10 | (100%) |
| Genipin - 0.1% | | |

Preparation of 30% w/w Poloxamer 407 in 1×PBS. (A) 1×PBS (Omnipur) is diluted from a 10× stock. 1 mM of erioglaucine (Sigma Aldrich) is added). F127 Pluronic® (Sigma Aldrich) powder is dissolved within the 1×PBS/1 mM erioglaucine. Optionally, at this step a contrasting agent such as iohexol (Histodenz, Sigma Aldrich) can be added. After the powder is dissolved it is refrigerated under agitation for 24 hours at a temperature <4° C. To obtain a sterile formulation in a 1 mL syringe transfer the refrigerated F127 solution to a pre-cooled syringe, obtain a cooled (<4° C.) 2 μm filter and cooled (<4° C.) receiving syringe. Connect the receiving syringe, filter, and dispensing syringe-slowly dispense the F127 solution to the receiving syringe through the filter. Additionally, the F127 can be sterilized via autoclave in which the F127 is placed in a glass vial, the autoclave is turned on at 126° C. for 20 minutes, after autoclaving place the solution in a fridge below the gelation temperature (ideally <4° C.) and aseptically transfer into a receiving syringe.

Preparation of thiolated Poly(vinyl alcohol) (TPVA). PVA is provided in a solution, dissolved in water or other solvents including but not limited to dimethyl sulfoxide (DMSO) or dimethylformamide (DMF). A thiol containing functional group such a 3-mercaptopropionic acid is added in the presence of an acid such as hydrogen chloride (HCl) while exposed to ambient temperatures >40° C. The resultant solution is then combined with Poly(ethylene glycol) diacrylate (PEGDa) resulting in crosslinking between the thiolated PVA and PEGDa. The resulting hydrogel can be injected in a pre-crosslinked form or configured to crosslink in-situ.

Preparation of Chitosan—PEGDa. Thiolated chitosan was dissolved in 1×PBS at double the desired final concentration. PEGDa was dissolved in 1×PBS and double the desired concentration. Two syringes were respectively filled with dissolved thiolated chitosan and PEGDa, the syringes were connected together and the solutions were mixed back and forth until adequately mixed in a single syringe. The chitosan and PEGDa crosslinked for 24 hours until ready for use.

Preparation of Chitosan—β-Glycerophosphate. Chitosan was dissolved in 0.14M HCl. β-Glycerophosphate was dissolved in water at double the desired concentration. The two solutions were refrigerated at 4° C. Upon stirring the chitosan solution, β-Glycerophosphate was added dropwise, the resulting mixture was stirred until homogenous. The final solution was drawn into a syringe until ready for use.

### Example 2: In-Vitro Evaluation of Polymer Formulations for Bulking Lung Tissue

In a similar experimental set-up to Example 1, a pair of excised pig lungs were attached to a ventilation machine which inflated the lungs to a constant pressure of 10 mmHg (slightly above the physiologic normal pleural pressure of −4 mmHg). The lungs were submerged in a water bath that was held at 37° C. to mimic physiologic body temperatures. A percutaneous coaxial delivery device as shown in FIGS. 3A-3F was used to deliver an array of different polymer formulations. The purpose of this experiment was to evaluate the efficacy of the polymer formulation, specifically the thermosensitive polymer formulation relative to the depth of insertion below the surface of the lung. The delivery device was inserted 0.5 cm into the lung and 0.5 mL of 30% w/w Poloxamer 407 with 1×PBS was injected with a polycarbonate syringe. Upon withdrawal of the device a further 0.5 mL of polymer was selectively deployed within the lung tissue as close to the surface as possible. It was evident that selectively administering and bulking polymer material closer to the lung surface (visceral pleura) resulted in higher sealing efficacy. The lungs were also excised and the shape of the implanted material was analyzed in which the shape was substantially circular, substantially elliptical right cylinder, a substantially circular cylinder, substantially elliptical oblique cylinder, a substantially circular truncated cone, and a substantially elliptical right truncated cone. The test was considered a success if no bubbles were visualized for 5 minutes. A summary of the results can be seen in Table 3 below.

TABLE 3

| | Polymer Formulation | Sealing Success |
|---|---|---|
| 1 mL, 0.5 cm depth | 30% Poloxamer 407 | 3/5 |
| | w/w in 1x PBS | |
| 0.5 mL 0.5 cm depth | 30% Poloxamer 407 | 5/5 |
| 0.5 mL, bulked near surface | w/w in 1x PBS | |

### Example 3: In-Vivo Feasibility Animal Trial: Lung Biopsy Experiments

The experiments described below were performed in 6 Yorkshire pigs under fluoroscopic imaging. The enhance the radiopacity of the polymer formulation a contrast agent was added. The final formulation used for the study was 27% w/w Poloxamer 407, 20% w/w Iohexol, and 0.09% w/w FD&C Blue #1 Dye (to assist with locating the material upon necropsy) this formulation had a gelation temp of 5.9° C. and an injection force of 14.6N when injected with a 1 ml syringe through second member 102 as described in FIGS. 3A-3F. It was realized that increasing iohexol concentration while maintaining Poloxamer 407 concentration (w/w) increased the injection force and increasing Poloxamer 407 concentration while maintaining iohexol concentration increased the injection force.

The protocol for the experiment was to use the assembled delivery device 100 wherein the second member 102 is placed within the first member 101 and the hub distal end 112 is coupled to the hub proximal end 106 and a syringe 108 containing the thermosensitive polymer formulation is coupled to the hub proximal end 113 as shown in FIG. 3B. The steps of the procedure are as follows: (a) the delivery device is primed so that the thermosensitive polymer formulation is seen coming out of the distal port (b) the syringe is removed from the hub proximal end 113 and the delivery device is inserted percutaneously into the pig along a path towards a target site until the tip of the delivery device is 0.5 cm within the lung parenchyma (the tip of the delivery device is 0.5 cm from the surface of the visceral pleura) (c) the syringe 108 is reattached and 0.5 mL of the thermosensitive polymer formulation is administered to the tract creating a bolus of material thereby providing an initial seal (d) the delivery device, optionally with the syringe attached or detached, is advanced until it reaches the target site (e) upon reaching the target site the second member 102 and optionally syringe 108 is removed while the first member 101 stays in place (f) an 18 gauge X cm length biopsy gun (C. R Bard, Franklin Lakes, NJ) is inserted through the first member 101 and 3 tissue samples are taken from the target site (optionally a lung ablation procedure can occur at this step wherein an ablation device is inserted through the first member 101) (g) upon completion of tissue sampling the second member 102 with syringe 108 attached is reinserted through the first member 101 and the device is retracted while the user selectively applies 0.5 mL of thermosensitive polymer material in the lung parenchymal tissue adjacent the visceral pleura. The user is able to determine the location of the delivery device to selectively apply the polymer material via intraoperative imaging, depth markers located on the delivery device, or a combination of both imaging and depth markers.

Upon full removal of the device an X-Ray was immediately taken verifying the location of the thermosensitive polymer had not migrated since initial injection. Additionally, another X-Ray was taken 4 hours later which also confirmed that the thermosensitive polymer was at the same location. The endotracheal tube was then removed and the pig was allowed to automatically breathe. 24 hours later the pig was re-intubated and another X-Ray was taken confirming the thermosensitive polymer was at the same location and had not fully degraded or migrated. The imaging verified that no pneumothorax or other clinically relevant event occurred during testing. A necropsy was then performed and slices of the tissue-polymer interface were taken which illustrated that no inflammatory, immunogenic responses, or tissue damage occurred.

### Example 4: In-Vivo Feasibility Animal Trial: CT-Imaging

Another experiment was performed evaluating the radiopacity of 30% w/w Poloxamer 407 material with 1×PBS without a contrast enhancing agent under CT-imaging. The purpose of this experiment was to evaluate whether the user could trace the in-vivo administration of the thermosensitive polymer formulation without a contrast enhancing agent. Similarly, a delivery device as described in FIGS. 3A-3F was used to inject the thermosensitive polymer formulation into the lung parenchyma of a pig under CT-imaging. Upon injection the thermosensitive polymer formulation was immediately apparent under CT-imaging.

### Example 5: Cohesiveness Evaluation

The thermosensitive polymers both described in Examples 3 and 4 where further evaluated for cohesivity. The delivery device was pulled after deploying the polymer and checked to see if any polymer or tissue was present on the device, no residual polymer or tissue was observed. Additionally, polymer was injected through the delivery device and the device was then set aside for 2 hours upon which further polymer was administered through the device, evaluating if the polymer dried or occluded within the device making subsequent injection harder. No occlusion or drying of the polymer was present after 2 hours and the injection force was the same.

### Example 6: Injectability Force of Polymers

A coaxial delivery device as described in FIGS. 3A-3F was used to deploy a polymer formulation through a 1 mL syringe which was attached to the tensile tips of an INSTRON device operating at a speed of 0.3 mL/s for a water filled control syringe injecting into air. The results are shown in Table 3 below which documents the peak injection force.

TABLE 4

| Polymer Formulation | Peak Injection Force (N) |
|---|---|
| Chitosan - Genipin | 110 |
| 30% w/w Poloxamer 407 in 1x PBS | 12.1 |
| 27% w/w Poloxamer 407, 20% Iohexol, | 15.1 |
| 1xPBS | |
| TPVA - 1.9% | 33 |
| PEGDa 3400 - 1.5% | |

### Example 7: In-Vivo Efficacy Animal Trial: CT-Imaging

The experiments described below were performed in 3 Yorkshire pigs under CT-imaging. Each experiment evaluated the efficacy of 30% w/w Poloxamer 407 material with 1×PBS without a contrast enhancing agent under CT-imaging. The purpose of this experiment was to evaluate the efficacy of the Poloxamer in preventing pneumothorax. The procedural protocol utilized is described in Example 3 above. Furthermore, CT images were taken pre-op, post-op, 2 hours, and 24 hours which allowed for evaluation of material migration degradation, and efficacy in preventing pneumothorax.

FIGS. 6A-6E details the experimental procedure in 1 of the 3 pigs. FIG. 6A shows the delivery device 100 advanced into the pig lung parenchyma. Once delivery device 100 is deployed into the lung parenchyma (within 2 cm of the visceral pleura), Poloxamer 407 is injected. After injection of Poloxamer 407, the needle is advanced further and an additional injection of Poloxamer 407 is performed, see FIG. 6B. After the second injection, the delivery device is advanced to the target site and biopsy samples are obtained. After the biopsy, the delivery device is retracted while injecting more Poloxamer 407. FIG. 6C shows the pig immediate post-procedure such that the Poloxamer 407 extends from the target site to the visceral pleura thereby protecting the pleural space from the invasion of air from the lung parenchyma. FIG. 6D shows the 2-hour timepoint which illustrates that the polymer is still in place and extends from the target site to the visceral pleura. FIG. 6E shows the 24-hour timepoint which illustrates that the majority of the material has resorbed and successfully protected the pig against pneumothorax.

Although ˜98% of pneumothoraces occur with 24 hours (Time-dependent analysis of incidence, risk factors and clinical significance of pneumothorax after percutaneous lung biopsy, Lim et al, European Society of Radiology, 2017), it may be desirable to decrease or extend the degradation of the material relative to the 24-hour timepoint. In order to decrease degradation time a lower % w/w of poloxamer may be used for example 25% w/w, 20% w/w, 15% w/w, or a range between 30% w/w and 0% w/w may be used. In order to increase the degradation, additional materials such as salts, polymers, and/or polysaccharides such as chitosan, hyaluronic acid, pectin, alginate, hydroxyethylcellulose, and methylcellulose may be combined with poloxamers and pluronics.

While certain embodiments have been described in terms of the preferred embodiments, it is understood that variations and modifications will occur to those skilled in the art. Therefore, it is intended that the appended claims cover all such equivalent variations that come within the scope of the following claims.

What is claimed is:

1. A medicament for use in the prevention and/or treatment of pneumothorax in a mammal by administering a first composition comprising a thermosensitive polymer to a group consisting of the pleural cavity, internal surface of the visceral pleura, external surface of the parietal pleural, internal surface of the parietal pleura, and/or within the lung parenchyma; wherein the first composition is located proximal to a target site.

2. The medicament of claim 1, wherein said thermosensitive polymer is a block polymer, random copolymer, graft polymer or branched copolymer.

3. The medicament of any preceding claim, wherein the first composition has a transition temperature between 5° C. and 40° C.

4. The medicament of any preceding claim, wherein the first composition comprises 5% to 40% by weight, preferably 20% to 35% by weight of said thermosensitive polymer.

5. The medicament of any preceding claim, wherein the first composition comprises 30% by weight of said thermosensitive polymer.

6. The medicament of claim 5 wherein, the first composition additionally comprises about 70% by weight of phosphate buffered saline.

7. The medicament of claim 1, wherein the thermosensitive polymer is selected from the group consisting of poloxamine 1107, poloxamine 1307, poloxamer 338 and poloxamer 407.

8. The medicament of claim 4, wherein the thermosensitive polymer is poloxamer 407.

9. The medicament of any preceding claim wherein the thermosensitive polymer is purified.

10. The medicament of claim 1, wherein the target site is selected from the group consisting of a hemorrhage, cancerous tissue, lesion, tumor, and organ.

11. The medicament of claim 1, wherein following administration, the distance between the first composition and the pleural cavity is less than the distance between the target site and the pleural cavity.

12. The medicament of claim 1, wherein administration of the thermosensitive polymer creates a tract in a tissue of a mammal, and, the thermosensitive polymer is administered in a gel state thereby temporarily occluding the tract.

13. The medicament of claim 1, where the first composition comprises a second composition optionally comprising a contrast-enhancing agent.

14. The medicament of claim 11, where said contrast-enhancing agent is selected from the group consisting of radiopaque materials, paramagnetic materials, heavy atoms, transition metals, lanthanides, actinides, dyes, and radionuclide-containing materials.

15. The medicament of any preceding claim, wherein said pneumothorax is caused by a device inserted into intercostal muscles, visceral pleura, parietal pleura, pleural pleura, or lung parenchyma, preferably by a device inserted into lung parenchyma, optionally wherein the device is selected from the group consisting of an access device, coaxial device, percutaneous delivery device, coaxial delivery device, biopsy device, and ablation device.

16. The medicament of claim 13, wherein administration of the thermosensitive polymer creates a tract in a tissue of a mammal, and, the thermosensitive polymer is administered at a proximal location in the tract before the device has reached a target site.

17. The medicament of any preceding claim, wherein said mammal is a human.

18. The medicament of any preceding claim, wherein the first composition, optionally further comprising the second composition, is injected using a syringe, optionally wherein the syringe has a volume of between about 1 milliliter to about 3 milliliters.

19. The medicament of claim 11, wherein the first composition, optionally further comprising the second composition, is injected using a delivery device;
wherein the delivery device comprises a first member, wherein the first member comprises a cannula and a hub, wherein the hub comprises a hub distal end, a hub proximal end, and ridges;
wherein the cannula is connected to the hub distal end;
the delivery device further comprising a second member, wherein the second member comprises a cannula, hub, port and sharp closed distal tip configured to pierce through tissue;
wherein the hub comprises a hub distal end, a hub proximal end, and grooves;
wherein the port is located on the sidewall of the cannula of the second member;
wherein the cannula of the second member is configured to connect to the distal end of the hub of the second member;
wherein the delivery device has an assembled position wherein the second member is inserted within the first member and the hub distal end of the second member is coupled to the hub proximal end of the first member;
wherein in the assembled position the cannula of the second member is concentric with the cannula of the first member;
wherein in the assembled position the port is located distal to the distal end of the cannula of the first member;
wherein a syringe can be optionally coupled to the hub proximal end of the second member;
wherein the syringe comprises the first composition and the first composition is configured to be injected from the syringe through the cannula of the second member and out of the distal port;
wherein the first composition comprises a thermosensitive polymer in a gel state.

20. The medicament of claim 19 wherein the hub proximal end of the second member is located at an angle relative to the cannula of the first and second members.

21. The medicament of claim 19 wherein the lumen extending through hub proximal end of the second member is located at an angle relative to the cannula of the first and second members.

22. A system for performing a medical procedure comprising: a delivery device, wherein the delivery device comprises a first member and optionally a second member;
wherein the delivery device has an assembled position wherein the second member extends through the first member and a hub distal end of the second member is coupled to a hub proximal end of the first member;
wherein the second member further comprises a cannula comprising a port on the sidewall;
wherein the port and cannula are fluidly connected to a syringe;
wherein the syringe is configured to deliver a thermosensitive gel in a gel state through the port to a target site;
wherein the second member further comprises a sharp closed distal tip configured to pierce through tissue;
wherein the second member is configured to be de-coupled a removed from the first member during a surgical procedure and the first member is configured to stay in place within a subject;
wherein the first member is configured to allow passage of a different device.

23. A medicament for use in the prevention and/or treatment of pneumothorax in a mammal by administering a material to a tract within a lung; wherein administration of the thermosensitive polymer causes the lung tissue to bulk; wherein the material is in a gel state and causes the lung tissue to remain bulked; and performing a surgical step in the tract.

24. The medicament of claim 23 wherein the surgical step is a lung biopsy.

25. The medicament of claim 23 wherein the thermosensitive polymer remains in the gel state.

26. The medicament of claim 23 wherein the thermosensitive polymer comprises poloxamer 407.

27. A method of administering a first composition at a target site comprising, introducing a delivery device into a tissue;
administering a first composition to the tissue;
advancing the delivery device through the first composition to a target site and performing a surgical step.

28. Use of a thermosensitive polymer for occluding a tract that is formed due to a percutaneous device, the use comprising;
advancing a delivery device to the lung parenchyma;
administering a first composition from a side port on the delivery device with the first composition forming a seal around the delivery device;
further advancing the delivery device through the first composition to a target site.

29. The use of claim 28, wherein the first composition comprises a thermosensitive polymer.

30. The use of claim 29, wherein the thermosensitive polymer is selected from the group consisting of poloxamine 1107, poloxamine 1307, poloxamer 338 and poloxamer 407.

31. The use of claim 30, wherein the thermosensitive polymer is poloxamer 407.

32. The use of claim 28 wherein the first composition comprises a liquid and gel state and the first composition is administered in a gel state.

33. A medical system for the prevention of pneumothorax during percutaneous biopsy, the medical system comprising, a delivery device configured to administer a first composition through a side port; a syringe configured to deliver a first composition in a gel state through the delivery device.

34. A medical system for the bulking of lung tissue, the medical system comprising, a delivery device configured to administer a first composition through a side port to lung parenchyma, a first composition configured to bulk lung tissue and prevent pneumothorax.

35. The medical system of claim 34 wherein the first composition comprises a thermosensitive polymer.

36. The medical system of claim 35, wherein the thermosensitive polymer is selected from the group consisting of poloxamine 1107, poloxamine 1307, poloxamer 338 and poloxamer 407.

37. The medical system of claim 36, wherein the thermosensitive polymer is poloxamer 407.

38. The medical system of claim 35 wherein the thermosensitive polymer is administered in a gel state.

39. A method for bulking mammalian tissue comprising the steps of: introducing a first composition to the mammalian tissue that can act as a bulking agent; the first composition comprising a thermosensitive polymer dissolved within a buffer, the thermosensitive polymer comprising about 30% w/w of the composition.

40. The method of claim 39 wherein the first composition is delivered via a coaxial delivery device.

41. The method of claim 40 wherein the first composition is delivered through a side port on the coaxial delivery device.

42. The method of claim 41 wherein the first composition is delivered to the lung parenchyma.

43. The method of claim 41 wherein the first composition is delivered adjacent to the visceral pleura.

44. The method of claim 39 wherein the first composition is delivered in a gel state and does not transform in-situ.

45. A method of occluding a tract in a mammal comprising the steps of:
administering into a tissue of a mammal, at or proximal to a target site, a composition comprising at least one thermosensitive polymer,
wherein: administering the thermosensitive polymer creates a tract in the tissue, and, the thermosensitive polymer is in a gel state and thereby immediately occluding the tract.

46. The method of claim 45, further comprising a surgical procedure.

47. The method of claim 45, wherein the thermosensitive polymer is a poloxamer or poloxamine.

48. The method of claim 45, wherein the at least one thermosensitive polymer is selected from the group consisting of poloxamer 407, poloxamer 338, poloxamer 118, poloxamine 1107 or poloxamine 1307.

49. The method of claim 45, wherein the at least one thermosensitive polymer is poloxamer 407.

50. The method of claim 45, wherein the shape of the composition in the tract is selected from the group consisting of a substantially circular, substantially elliptical right cylinder, a substantially circular cylinder, substantially elliptical oblique cylinder, a substantially circular truncated cone, and a substantially elliptical right truncated cone.

51. The method of claim 50, wherein said composition comprises at least one thermosensitive polymer selected from the group consisting of a block copolymer, random copolymer, graft polymer, branched copolymer, or a combination thereof.

52. The method of claim 50, wherein said at least one thermosensitive polymer is poloxamer 407.

53. The method of claim 50, wherein said wherein said composition has a transition temperature of between about 5° C. and 15° C.

54. The method of claim 45, wherein said tissue is selected from the group consisting of visceral pleura, parietal pleura, pleural cavity, lung parenchyma, kidney, and liver.

55. The method of claim 46, wherein the surgical procedure is selected from the group consisting of a lung biopsy, a percutaneous lung biopsy, a transthoracic lung biopsy, a lung ablation, a percutaneous lung ablation, and transthoracic lung ablation.

56. The method of claim 50, wherein the composition in the tract reduces complications selected from the group consisting of bleeding, hemorrhage, pneumothorax, pulmonary air embolism, and tumor tract seeding.

57. The method of claim 49, wherein said target site is selected from the group consisting of a hemorrhage, cancerous tissue, tumor, and organ.

58. The method of claim 49, wherein said composition occludes said tract for greater than 24 hours.

59. The method of claim 49, wherein said composition occludes said tract for at least 4 hours.

60. The method of claim 49, wherein said composition is introduced to said tissue via a device selected from the group consisting of a percutaneous access device, coaxial access device, needle, and catheter.

61. The method of claim 60, wherein said coaxial access device comprises a first member, wherein the first member comprises a cannula and a hub, wherein the hub comprises a hub distal end, a hub proximal end, and ridges;
wherein the cannula is connected to the hub distal end;
the delivery device further comprising a second member, wherein the second member comprises a cannula, hub, and port;
wherein the hub comprises a hub distal end, a hub proximal end, and grooves;
wherein the port is located on the sidewall of the cannula of the second member;
wherein the cannula of the second member is configured to connect to the distal end of the hub of the second member;
wherein the delivery device has an assembled position wherein the second member is inserted within the first member and the hub distal end of the second member is coupled to the hub proximal end of the first member;
wherein in the assembled position the cannula of the second member is concentric with the cannula of the first member;
wherein in the assembled position the port is located distal to the distal end of the cannula of the first member;
wherein a syringe can be optionally coupled to the hub proximal end of the second member;
wherein the syringe comprises the first composition and the first composition is configured to be injected from the syringe through the cannula of the second member and out of the distal port.

62. The method of claim 61 wherein the proximal hub of the second member is located at an angle relative to the cannula of the first member and second member.

63. The method of claim 49, wherein said composition is administered to said tissue using a syringe.

64. The method of claim 49, wherein said composition is administered to said tissue using a syringe with a volume of between about 0.1 milliliters to 10 milliliters.

65. The method of claim 64, wherein said volume is between 1 milliliter to 3 milliliters.

66. A method for preventing pneumothorax comprising:
inserting a delivery device into lung parenchyma;
administering a first composition in a sufficient volume to bulk the lung parenchyma.

67. The method of claim 66 wherein the delivery device is a coaxial delivery device.

68. The method of claim 66 wherein the first composition is a thermosensitive polymer.

69. The method of claim 68 wherein the thermosensitive polymer is selected from the group consisting of poloxamer 407, poloxamer 338, poloxamer 118, poloxamine 1107 or poloxamine 1307.

70. The method of claim 69 wherein the thermosensitive polymer is dissolved in a carrier resulting in an injectable composition.

71. The method of claim 70 wherein the carrier comprises a phosphate buffer.

72. The method of claim 69 wherein the thermosensitive polymer comprises poloxamer 407.

73. The method of claim 72 wherein poloxamer 407 comprises about 30% w/w of the composition.

74. The method of claim 67 wherein a syringe containing the first composition is fixedly attached to the coaxial delivery device.

75. The method of claim 67 wherein the coaxial delivery device comprises a first member having a cannula, a port, and closed sharp distal tip configured to pierce through tissue.

76. The method of claim 75 wherein the first member has a proximal hub that has a axis therethrough that is located at an off-angle relative to the cannula.

77. The method of claim 75 wherein the coaxial delivery device comprises a second member comprising a cannula that allows passage therethrough of the first member.

78. The method of claim 76 wherein the coaxial delivery device comprises an assembled position wherein the first member is housed within and coupled to the second member.

79. The method of claim 66 wherein after administering a first composition the delivery device travels through the first composition to a target site.

80. The method of claim 75, wherein the closed sharp distal tip is located distal to the first composition when the first composition is administered.

81. The method of claim 66 wherein the first composition is configured to form a seal around the delivery device when administered.

82. The method of claim 66 wherein the first composition is injected in a gel state and does not transform in-situ.

83. The method of claim 68 wherein the first composition is injected in a gel state and does not transform in-situ.

84. A device comprising:
a cannula ending in a closed sharp distal tip configured to pierce through tissue;
a proximal hub comprising a distal end and proximal end;
the distal end of the proximal hub configured to couple to the proximal end of the cannula;
the hub proximal end having an off angle relative to the cannula.

85. The device of claim 84 wherein the cannula contains a side port.

86. The device of claim 85 wherein the side port is located on the distal end of the device.

87. The device of claim 86 wherein the side port is fluidly connected to the proximal hub.

88. The device of claim 84 wherein the hub proximal end is configured to couple to a syringe.

89. A method for reducing or preventing complications during lung biopsy comprising:
administering to the lung parenchyma a first composition comprising a thermosensitive polymer with a delivery device.

90. The method of claim 89, wherein the thermosensitive polymer is selected from the group consisting of poloxamer 407, poloxamer 338, poloxamer 118, poloxamine 1107 or poloxamine 1307.

91. The method of claim 90, wherein the thermosensitive polymer comprises poloxamer 407.

92. The method of claim 91, wherein the thermosensitive polymer is dissolved in a buffer that creates an injectable material.

93. The method of claim 92, wherein the buffer is a phosphate buffer.

94. The method of claim 91, wherein the thermosensitive polymer comprises 30% w/w of poloxamer 407, wherein the composition optionally comprises at least 0.05% w/w of a dye.

95. The method of claim 94 wherein the dye comprises FD&C Blue.

96. The method of claim 91, wherein the first composition is administered in a gel state and does not transform in-situ.

97. The method of claim 89, wherein the thermosensitive polymer is administered adjacent to the visceral pleura.

98. The method of claim 89, wherein the delivery device is inserted towards a target site, wherein the delivery device is configured to deliver the first composition at a location proximal to the target site before the target site has been reached.

99. The method of claim 97, wherein the delivery device forms a tract, upon administering the first composition the tract becomes occluded.

100. The method of claim 98, wherein the delivery device has a closed sharp distal end configured to pierce through tissue, a cannula, a hub, and a port located on the sidewall of the cannula.

101. The method of claim 99, wherein the hub comprises a hub proximal end that can couple to a syringe wherein the syringe can deliver the first composition from the syringe through the cannula, through the port, and to the tract.

102. The method of claim 101 wherein the syringe comprises a volume between about 1 ml to 3 ml.

103. The method of claim 100, wherein the first composition is injected in a gel state and the first composition does not transform in-situ.

104. The method of claim 100 wherein the first composition forms around the entire outer cannula of the delivery device thereby forming a seal.

105. The method of claim 100 wherein the closed sharp distal end is located distal to the first composition when the first composition is administered.

106. The method of claim 100 wherein after the first composition is administered the delivery device is advanced to the target site and the first composition stays in place.

107. The method of claim 104 wherein the first composition stays within the tract for at least 4 hours.

108. The method of claim 100 wherein after administering the first composition the first composition does not occlude the cannula of the delivery device.

109. A medicament for preventing complications during percutaneous transthoracic procedures comprising: advancing a delivery device to lung parenchyma wherein the advancement of the delivery device creates a void in the lung parenchyma; administering a first composition comprising a thermosensitive polymer through the delivery device and filling the void.

110. The medicament of claim 109 wherein the thermosensitive polymer is selected from the group consisting of poloxamer 407, poloxamer 338, poloxamer 118, poloxamine 1107, and poloxamine 1307.

111. The medicament of claim 110, wherein the thermosensitive polymer comprises poloxamer 407.

112. The medicament of claim 109, wherein the first composition comprises between about 10% to 40% w/w of the thermosensitive polymer.

113. The medicament of claim 109, wherein the thermosensitive polymer is delivered in a gel state and does not transform in-situ.

114. The medicament of claim 109, wherein the first composition is delivered through a side port located on the delivery device.

115. The medicament of claim 114, wherein the side port is located proximal to a sharp closed distal end of the delivery device.

116. The medicament of claim 109, wherein the first composition fills the void for at least 4 hours.

117. The medicament of claim 109, wherein the first composition is biodegradable.

118. A medicament for use in the prevention and/or treatment of pneumothorax in a mammal by administering a first composition comprising a thermosensitive polymer to a group consisting of the pleural cavity, internal surface of the visceral pleura, external surface of the parietal pleural, internal surface of the parietal pleura, and/or within the lung parenchyma; wherein the first composition is located proximal to a target site.

119. The medicament of claim 118, wherein said thermosensitive polymer is a block polymer, random copolymer, graft polymer or branched copolymer.

120. The medicament of any preceding claim, wherein the first composition has a transition temperature between 5° C. and 40° C.

121. The medicament of any preceding claim, wherein the first composition comprises 5% to 40% by weight, preferably 20% to 35% by weight of said thermosensitive polymer.

122. The medicament of any preceding claim, wherein the first composition comprises 30% by weight of said thermosensitive polymer.

123. The medicament of any preceding claim wherein the thermosensitive polymer is purified.

124. The medicament of claim 118, wherein the target site is selected from the group consisting of a hemorrhage, cancerous tissue, lesion, tumor, and organ.

125. The medicament of claim 118, wherein following administration, the distance between the first composition and the pleural cavity is less than the distance between the target site and the pleural cavity.

126. The medicament of claim 125, wherein, following administration, the distance between the first composition and the pleural cavity is less than 2 centimeters.

127. The medicament of claim 118, wherein administration of the thermosensitive polymer creates a tract in a tissue of a mammal, and, the thermosensitive polymer is administered in a gel state thereby temporarily occluding the tract.

128. The medicament of claim 127, wherein the thermosensitive polymer is administered via a coaxial device.

129. The medicament of claim 127, wherein the thermosensitive polymer degrades between 2 hours and 2 weeks.

130. The medicament of claim 129, wherein the thermosensitive polymer is fully degraded within 24 hours.
